SEQUENCE LISTING (34 sequences)

SEQ ID NO: 1 - protein, 14 aa, synthetic construct
AGWENQAGELKKIL

SEQ ID NO: 2 - protein, 14 aa, synthetic construct
SGPRPTCHLDRHSS

SEQ ID NO: 3 - protein, 14 aa, synthetic construct
WKRQEEICPKAFGG

SEQ ID NO: 4 - protein, 14 aa, synthetic construct
RGACCGAPALGGAF

SEQ ID NO: 5 - protein, 14 aa, synthetic construct
HRLSPQMTTVQKAA

SEQ ID NO: 6 - protein, 14 aa, synthetic construct
KVRLSLSSRTEGEA

SEQ ID NO: 7 - protein, 14 aa, synthetic construct
LGNKPSYSEEAGVR

SEQ ID NO: 8 - protein, 14 aa, synthetic construct
WAGGSHMRTHMDIM

SEQ ID NO: 9 - protein, 14 aa, synthetic construct
WSRREGSGVPPPPP

SEQ ID NO: 10 - protein, 12 aa, synthetic construct
RGLPRPSCRDQA

SEQ ID NO: 11 - protein, 14 aa, synthetic construct
WAGDGFKICGQRVH

SEQ ID NO: 12 - protein, 14 aa, synthetic construct
WAGPVFSQRPPLGQ

SEQ ID NO: 13 - protein, 14 aa, synthetic construct
WAPGSFCKDSHSRS

SEQ ID NO: 14 - protein, 14 aa, synthetic construct
PRGPPFMDPWPVAA

SEQ ID NO: 15 - protein, 14 aa, synthetic construct
AGPGCASDYLQPSR

SEQ ID NO: 16 - protein, 14 aa, synthetic construct
CGTWGRPEREVSDG

SEQ ID NO: 17 - protein, 14 aa, synthetic construct
MGPQACSAGLETAT

SEQ ID NO: 18 - protein, 43 aa, synthetic construct
AGWENQAGELKKILNTGPGSQELMVFSWARPLCWACVGTRTSR

SEQ ID NO: 19 - protein, 45 aa, synthetic construct
SGPRPTCHLDRHSSLHIPPGPRHPQVNFLSYILLRVCVLMRGASA

SEQ ID NO: 20 - protein, 47 aa, synthetic construct
THNEKEVNMQVETGVMWPQARRHLRPRSWKRQEEICPKAFGGGSMAL

SEQ ID NO: 21 - protein, 65 aa, synthetic construct
RGACCGAPALGGAFLLLPPPTGRRKGVYEGSLRGSIRAAALWLPDVSESVFRLYTTSFFFFLIYL

SEQ ID NO: 22 - protein, 26 aa, synthetic construct
EFCYEEHNVHILHRLSPQMTTVQKAA

SEQ ID NO: 23 - protein, 40 aa, synthetic construct
KVRLSLSSRTEGEAGAVRGVPGSTTPGLHPLIVTLSGTVP

SEQ ID NO: 24 - protein, 65 aa, synthetic construct
QPRPARRVVPRETAAGAWGRALGRPCVSLGNKPSYSEEAGVRKDPPPLELVVAGLPCDRSSSCPR

SEQ ID NO: 25 - protein, 25 aa, synthetic construct
VGTGWTMQTCSWAGGSHMRTHMDIM

SEQ ID NO: 26 - protein, 78 aa, synthetic construct
PGAGRRARWVTRRRGAHLSEPRRPPAKWWSRREGSGVPPPPPEDLGEAQDRPRTAARLLRKLKRVNVSEVRGAASVSL

SEQ ID NO: 27 - protein, 12 aa, synthetic construct
RGLPRPSCRDQA

SEQ ID NO: 28 - protein, 32 aa, synthetic construct
PVPGPYVPFSPVLLWAGDGFKICGQRVHLCSP

SEQ ID NO: 29 - protein, 33 aa, synthetic construct
SNKGKPPSGRQLRAWAGPVFSQRPPLGQRRVPS

SEQ ID NO: 30 - protein, 21 aa, synthetic construct
YFLLIHAWAPGSFCKDSHSRS

SEQ ID NO: 31 - protein, 20 aa, synthetic construct
RAPSQSPRGPPFMDPWPVAA

SEQ ID NO: 32 - protein, 43 aa, synthetic construct
GGATGQRGMASSRAVGHGGQWGMVGSGAAGPGCASDYLQPSRT

SEQ ID NO: 33 - protein, 20 aa, synthetic construct
APGGAACGTWGRPEREVSDG

SEQ ID NO: 34 - protein, 42 aa, synthetic construct
GKPRGPVWGGRPWGPMAGGGRTALVPGDMGPQACSAGLETAT

(19) United States
(12) Patent Application Publication
(10) Pub. No.: US 2026/0273034 A1
(43) Pub. Date: Sep. 17, 2026

(54) NON-CODING RNA PEPTIDE ANTIGENS FOR CANCER VACCINES, THERAPEUTICS, AND DIAGNOSTICS

(71) Applicant: Calviri, Inc., Phoenix, AZ (US)

(72) Inventor: Stephen Albert Johnston, Phoenix, AZ (US)

(21) Appl. No.: 19/469,099

(22) PCT Filed: Mar. 27, 2024
(86) PCT No.: PCT/US2024/021622

§ 371 Date: Sep. 25, 2025

Related U.S. Application Data
(60) Provisional application No. 63/492,907, filed on Mar. 29, 2023.

(51) Int. Cl. A61K 39/00; A61P 35/00; A61P 37/04; C07K 16/30

(52) U.S. Cl. CPC A61K 39/0011; A61P 35/00; A61P 37/04; C07K 16/30; A61K 2039/552

(57) ABSTRACT

Provided herein are methods of identifying ncRNA neoantigens for diagnosing, treating, and preventing cancer. Also disclosed are methods and compositions for administering identified ncRNA neoantigens for the treatment and prevention of cancer.

### INCORPORATION BY REFERENCE TO ANY PRIORITY APPLICATIONS

This application claims priority to U.S. Provisional Patent Application No. 63/492,907, filed Mar. 29, 2023, which is hereby incorporated by reference in its entirety. Any and all applications for which a foreign or domestic priority claim is identified in the Application Data Sheet as filed with the present application are hereby incorporated by reference under 37 CFR 1.57.

### REFERENCE TO SEQUENCE LISTING

The present application is filed with a Sequence Listing in Electronic format. The Sequence Listing is provided as a file entitled “SEQLISTING_CALV045WO,” created Mar. 25, 2024, which is approximately 30.2 kb in size. The information in the electronic format of the sequence listing is incorporated herein by reference in its entirety.

### BACKGROUND

Checkpoint inhibitor immunotherapeutics are revolutionizing cancer therapy. However, even in the most responsive cancers, a substantial portion (50%-80%) of the patients have poor to no positive response. The evidence to date is that whether a patient has an effective response to the treatment depends on the nature of the immune response they have established against the tumor. More specifically, the level and quality of the immune response to neoantigens in the cancer seems to be most important.

### SUMMARY

Embodiments provided herein relate to systems, compositions, and methods that include neoantigens encoded by non-coding RNAs (ncRNA). In particular, the systems, compositions, and methods are used for detecting cancer, for predicting response to immune therapy, and/or for creating preventative and therapeutic vaccines, and arrays that include ncRNAs that may be used in these methods. Also provided herein are methods for treating or preventing cancer by administration of a vaccine that includes one or more ncRNA peptides or one or more nucleic acid sequences encoding one or more ncRNA peptides. Some embodiments relate to methods of identifying ncRNA peptides (ncRNAPs) that are immunogenic, cancer-specific, and/or shared across cancers. Also provided herein are methods of treating or preventing cancer by administration of a therapeutic molecule that binds to a ncRNAP.

Some embodiments provided herein relate to methods of identifying one or more ncRNA peptides (ncRNAPs) that are immunogenic, cancer-specific, and/or shared across cancers. In some embodiments, the methods include contacting a ncRNA peptide array including a plurality of ncRNAPs with a first biological sample obtained from a first individual known to have a cancer, measuring binding of the first biological sample to the ncRNA peptide array, contacting the same type of ncRNA peptide array with a second biological sample obtained from a second individual, measuring binding of the second biological sample to the ncRNA peptide array, comparing the binding of the two biological samples to the ncRNA peptide array, and identifying one or more ncRNAPs that are immunogenic, cancer-specific, and/or shared across cancers. In some embodiments, measuring binding includes detecting antibody reactivity to the plurality of ncRNAPs. In some embodiments, the second individual is a control individual without cancer. In some embodiments, the second individual is also known to have a cancer. In some embodiments, the second individual is known to have the cancer of the first individual. In some embodiments, the second individual and the first individual have different types of cancer. In some embodiments, the methods further include determining a nucleic acid sequence of said ncRNAPs that are immunogenic to identify one or more nucleic acids encoding ncRNAPs that are immunogenic, cancer-specific, and/or shared across cancers. In some embodiments, the array comprises at least one peptide having at least 95% sequence identity with any of SEQ ID NOs: 1-17. In some embodiments, the ncRNA peptide array includes at least about 10, about 100, about 200, about 300, about 500, about 1,000, about 2,000, about 3,000, about 5,000, about 7,500, about 10,000, about 12,500, about 15,000, about 17,500, about 20,000, about 22,500, about 25,000, about 27,500, about 30,000, about 32,500, about 35,000, about 37,500, about 40,000, about 50,000, about 100,000, about 200,000, about 300,000, about 400,000, about 500,000, about 600,000, about 700,000, about 800,000, about 900,000, about 1,000,000, about 1,500,000, about 2,000,000, about 2,500,000, about 3,000,000, about 3,500,000, about 4,000,000, about 4,500,000, or about 5,000,000 ncRNAPs. In some embodiments, the plurality of ncRNAPs is in-situ synthesized on the array. In some embodiments, the plurality of ncRNAPs is fixed on a substrate. In some embodiments, the substrate includes glass, silica, composite, resin, or combination thereof. In some embodiments, binding can be detected by at least one of fluorescence, luminescence, calorimetry, chromatography, radioactivity, Bio-Layer Interferometry, electro-interference sensing, or surface plasmon resonance.

In some embodiments, disclosed herein are methods of measuring an immune response to a plurality of ncRNA peptides. In some embodiments, the method includes a) contacting a biological sample with an ncRNA peptide array comprising a plurality of ncRNA peptides, and b) measuring binding of the biological sample to the ncRNA peptide array. In some embodiments, the ncRNA peptide array comprises at least one peptide having at least 95% sequence identity with any of SEQ ID NOs: 1-17. In some embodiments, measuring binding comprises detecting antibody reactivity to the plurality of ncRNAPs. In some embodiments, the ncRNA peptide array comprises at least about 10, about 100, about 200, about 300, about 500, about 1,000, about 2,000, about 3,000, about 5,000, about 7,500, about 10,000, about 12,500, about 15,000, about 17,500, about 20,000, about 22,500, about 25,000, about 27,500, about 30,000, about 32,500, about 35,000, about 37,500, about 40,000, about 50,000, about 100,000, about 200,000, about 300,000, about 400,000, about 500,000, about 600,000, about 700,000, about 800,000, about 900,000, about 1,000,000, about 1,500,000, about 2,000,000, about 2,500,000, about 3,000,000, about 3,500,000, about 4,000,000, about 4,500,000, or about 5,000,000 ncRNAPs. In some embodiments, the plurality of ncRNAPs is in-situ synthesized on the array. In some embodiments, the plurality of ncRNAPs is fixed on substrate. In some embodiments, the substrate comprises glass, silica, composite, resin, or combination thereof. In some embodiments, binding is detected by at least one of fluorescence, luminescence, calorimetry, chromatography, radioactivity, Bio-Layer Interferometry, electro-interference sensing, or surface plasmon resonance.

In some embodiments, disclosed herein are arrays. In some embodiments, the arrays include a plurality of ncRNA peptides. In some embodiments, the array comprises at least one peptide having at least 95% sequence identity with any of SEQ ID NOs: 1-17. In some embodiments, the ncRNAPs on the array are spaced between 3 and 9 μm. In some embodiments, the arrays include about 10, about 100, about 200, about 300, about 500, about 1,000, about 2,000, about 3,000, about 5,000, about 7,500, about 10,000, about 12,500, about 15,000, about 17,500, about 20,000, about 22,500, about 25,000, about 27,500, about 30,000, about 32,500, about 35,000, about 37,500, about 40,000, about 50,000, about 100,000, about 200,000, about 300,000, about 400,000, about 500,000, about 600,000, about 700,000, about 800,000, about 900,000, about 1,000,000, about 1,500,000, about 2,000,000, about 2,500,000, about 3,000,000, about 3,500,000, about 4,000,000, about 4,500,000, or about 5,000,000 ncRNAPs. In some embodiments, the plurality of predicted potential encoded ncRNA peptides are in-situ synthesized on the array. In some embodiments, the plurality of ncRNA peptides is in-situ synthesized on the array. In some embodiments, the plurality of ncRNA peptides is fixed on a substrate. In some embodiments, the substrate includes glass, silica, composite, resin, or combination thereof. In some embodiments, the array is configured to detect binding by at least one of fluorescence, luminescence, calorimetry, chromatography, radioactivity, Bio-Layer Interferometry, electro-interference sensing, or surface plasmon resonance. In some embodiments, the plurality of ncRNA peptides include two or more pooled ncRNAPs.

Some embodiments provided herein relate to methods of detecting cancer in a subject. In some embodiments, the methods include obtaining a biological sample from a subject, contacting the biological sample from the subject with an array including a plurality of ncRNAPs resulting from non-conventional translation of a ncRNA, measuring binding of the biological sample to the plurality of ncRNAPs, and analyzing the binding to predict whether the subject has cancer. In some embodiments, measuring binding includes detecting antibody reactivity to at least one peptide of the array. In some embodiments, the plurality of ncRNAPs is fixed on a substrate. In some embodiments, the substrate includes glass, silica, composite, resin, or combination thereof. In some embodiments, the plurality of ncRNAPs is in-situ synthesized on the array. In some embodiments, binding is detected by at least one of fluorescence, luminescence, calorimetry, chromatography, radioactivity, electro-interference, Bio-Layer Interferometry, electro-interference sensing, or surface plasmon resonance. In some embodiments, the array comprises at least one peptide having at least 95% sequence identity with any of SEQ ID NOs: 1-17. In some embodiments, the array includes about 10, about 100, about 200, about 300, about 500, about 1,000, about 2,000, about 3,000, about 5,000, about 7,500, about 10,000, about 12,500, about 15,000, about 17,500, about 20,000, about 22,500, about 25,000, about 27,500, about 30,000, about 32,500, about 35,000, about 37,500, about 40,000, about 50,000, about 100,000, about 200,000, about 300,000, about 400,000, about 500,000, about 600,000, about 700,000, about 800,000, about 900,000, about 1,000,000, about 1,500,000, about 2,000,000, about 2,500,000, about 3,000,000, about 3,500,000, about 4,000,000, about 4,500,000, or about 5,000,000 ncRNAPs. In some embodiments, the biological sample includes blood, serum, plasma, cerebrospinal fluid, saliva, urine, or combinations thereof. In some embodiments, the biological sample includes antibodies. In some embodiments, the subject is a mammal. In some embodiments, the subject is a human, a dog, a cat, a mouse, a rat, a rabbit, a horse, a cow, or a pig. In some embodiments, the subject is suspected of having a cancer. In some embodiments, the cancer is selected from the group consisting of acute lymphoblastic leukemia, acute monocytic leukemia, acute myeloid leukemia, acute promyelocytic leukemia, adenocarcinoma, adult T-cell leukemia, astrocytoma, bladder cancer, bone cancer, brain tumor, breast cancer, Burkitt's lymphoma, carcinoma, cervical cancer, chronic lymphocytic leukemia, chronic myelogenous leukemia, colon cancer, colorectal cancer, endometrial cancer, glioblastoma multiforme, glioma, hepatocellular carcinoma, Hodgkin's lymphoma, inflammatory breast cancer, kidney cancer, leukemia, lung cancer, lymphoma, Hodgkin lymphoma, non-small cell lung cancer, ovarian cancer, pancreatic cancer, pituitary tumor, prostate cancer, retinoblastoma, skin cancer, small cell lung cancer, squamous cell carcinoma, stomach cancer, T-cell leukemia, T-cell lymphoma, thyroid cancer, and Wilms' tumor.

Some embodiments provided herein relate to methods of predicting the response of a subject with cancer to an immunotherapy. In some embodiments, the methods include obtaining a biological sample from a subject with cancer, contacting the biological sample to an array including a plurality of ncRNAPs, and measuring binding of the biological sample to the plurality of ncRNAPs. In some embodiments, measuring binding includes detecting antibody reactivity to at least one peptide of the array. In some embodiments, the methods further include analyzing the binding to predict whether immunotherapy would be effective in treating the subject's cancer. In some embodiments, the methods further include analyzing the binding to predict whether immunotherapy would elicit an adverse immune response to immunotherapy in the subject. In some embodiments, analyzing the binding includes comparing the binding of the biological sample to binding of another subject who responded positively to immunotherapy or experienced an adverse immune response in response to immunotherapy. In some embodiments, the plurality of ncRNAPs is fixed on a substrate. In some embodiments, the substrate includes glass, silica, composite, resin, or combination thereof. In some embodiments, the plurality of ncRNAPs is in-situ synthesized on the array. In some embodiments, binding is detected by at least one of fluorescence, luminescence, calorimetry, chromatography, radioactivity, electro-interference, Bio-Layer Interferometry, electro-interference sensing, or surface plasmon resonance. In some embodiments, the array comprises at least one peptide having at least 95% sequence identity with any of SEQ ID NOs: 1-17. In some embodiments, the array includes at least about 10, about 100, about 200, about 300, about 500, about 1,000, about 2,000, about 3,000, about 5,000, about 7,500, about 10,000, about 12,500, about 15,000, about 17,500, about 20,000, about 22,500, about 25,000, about 27,500, about 30,000, about 32,500, about 35,000, about 37,500, about 40,000, about 50,000, about 100,000, about 200,000, about 300,000, about 400,000, about 500,000, about 600,000, about 700,000, about 800,000, about 900,000, about 1,000,000, about 1,500,000, about 2,000,000, about 2,500,000, about 3,000,000, about 3,500,000, about 4,000,000, about 4,500,000, or about 5,000,000 ncRNAPs. In some embodiments, the biological sample includes blood, serum, plasma, cerebrospinal fluid, saliva, urine, or combinations thereof. In some embodiments, the biological sample includes antibodies. In some embodiments, the subject is a mammal. In some embodiments, the subject is a human, a dog, a cat, a mouse, a rat, a rabbit, a horse, a cow, or a pig. In some embodiments, the cancer is selected from the group consisting of acute lymphoblastic leukemia, acute monocytic leukemia, acute myeloid leukemia, acute promyelocytic leukemia, adenocarcinoma, adult T-cell leukemia, astrocytoma, bladder cancer, bone cancer, brain tumor, breast cancer, Burkitt's lymphoma, carcinoma, cervical cancer, chronic lymphocytic leukemia, chronic myelogenous leukemia, colon cancer, colorectal cancer, endometrial cancer, glioblastoma multiforme, glioma, hepatocellular carcinoma, Hodgkin's lymphoma, inflammatory breast cancer, kidney cancer, leukemia, lung cancer, lymphoma, malignant mesothelioma, medulloblastoma, melanoma, multiple myeloma, neuroblastoma, non-Hodgkin lymphoma, non-small cell lung cancer, ovarian cancer, pancreatic cancer, pituitary tumor, prostate cancer, retinoblastoma, skin cancer, small cell lung cancer, squamous cell carcinoma, stomach cancer, T-cell leukemia, T-cell lymphoma, thyroid cancer, and Wilms' tumor.

Some embodiments provided herein relate to vaccine compositions. In some embodiments, the vaccine compositions include one or more ncRNA peptides. In some embodiments, the vaccine compositions further include an adjuvant. In some embodiments, the adjuvant is ABM2, AS01B, AS02, AS02A, Adjumer, Adjuvax, Algammulin, alum, aluminum phosphate, aluminum potassium sulfate, Bordetella pertussis, calcitriol, chitosan, cholera toxin, CpG, dibutyl phthalate, dimethyldioctadecylammonium bromide (DDA), Freund's adjuvant, Freund's complete, Freund's incomplete (IFA), GM-CSF, GMDP, gamma inulin, glycerol, HBSS (Hank's Balanced Salt Solution), polyinosinic-polycytidylic acid stabilized with polylysine and carboxymethylcellulose (poly-ICLC, also known as Hiltonol), IL-12, IL-2, imiquimod, interferon-gamma, ISCOM, lipid core peptide (LCP), lipofectin, lipopolysaccharide (LPS), liposomes, MF59, MLP+TDM, monophosphoryl lipid A, Montanide IMS-1313, Montanide ISA 206, Montanide ISA 720, Montanide ISA-51, Montanide ISA-50, nor-MDP, oil-in-water emulsion, P1005 (non-ionic copolymer), Pam3Cys (lipoprotein), pertussis toxin, poloxamer, QS21, RaLPS, Ribi, saponin, Seppic ISA 720, soybean oil, squalene, Syntex adjuvant formulation (SAF), synthetic polynucleotides (poly IC/poly AU), TiterMax tomatine, Vaxfectin, XtendIII, or Zymosan.

Some embodiments herein relate to methods of designing a cancer vaccine. In some embodiments, the methods include identifying one or more ncRNA peptides (ncRNAPs) or one or more nucleic acids encoding ncRNAPs that are immunogenic and preparing a vaccine including one or more ncRNAPs or one or more nucleic acids encoding ncRNAPs that are immunogenic. In some embodiments, identifying one or more ncRNAPs or one or more nucleic acids encoding ncRNAPs that are immunogenic includes contacting a ncRNA peptide array including one or more ncRNAPs with a first biological sample obtained from a first individual known to have a cancer, measuring binding of the first biological sample to the ncRNA peptide array, contacting the same type of ncRNA peptide array with a second biological sample obtained from a second individual, measuring binding of the second biological sample to the ncRNA peptide array, comparing the binding of the two biological samples to the ncRNA peptide array, and identifying one or more ncRNAPs that are immunogenic. In some embodiments, the methods further include determining a nucleic acid sequence of said ncRNAPs that are immunogenic to identify one or more nucleic acids encoding ncRNAPs that are immunogenic. In some embodiments, measuring binding includes detecting antibody reactivity to the plurality of ncRNAPs. In some embodiments, the second individual is a control individual without cancer. In some embodiments, the second individual is also known to have a cancer. In some embodiments, the second individual is known to have the cancer of the first individual. In some embodiments, the second individual and the first individual have different types of cancer. In some embodiments, the ncRNA peptide array comprises at least one peptide having at least 95% sequence identity with any of SEQ ID NOs: 1-17. In some embodiments, the ncRNA peptide array includes at least about 10, about 100, about 200, about 300, about 500, about 1,000, about 2,000, about 3,000, about 5,000, about 7,500, about 10,000, about 12,500, about 15,000, about 17,500, about 20,000, about 22,500, about 25,000, about 27,500, about 30,000, about 32,500, about 35,000, about 37,500, about 40,000, about 50,000, about 100,000, about 200,000, about 300,000, about 400,000, about 500,000, about 600,000, about 700,000, about 800,000, about 900,000, about 1,000,000, about 1,500,000, about 2,000,000, about 2,500,000, about 3,000,000, about 3,500,000, about 4,000,000, about 4,500,000, or about 5,000,000 ncRNAPs. In some embodiments, the plurality of ncRNAPs is in-situ synthesized on the array. In some embodiments, the plurality of ncRNAPs is fixed on a substrate. In some embodiments, the substrate includes glass, silica, composite, resin, or combination thereof. In some embodiments, binding is detected by at least one of fluorescence, luminescence, calorimetry, chromatography, radioactivity, electro-interference, Bio-Layer Interferometry, electro-interference sensing, or surface plasmon resonance. In some embodiments, the vaccine compositions further include an adjuvant. In some embodiments, the adjuvant is ABM2, AS01B, AS02, AS02A, Adjumer, Adjuvax, Algammulin, Alum, Aluminum phosphate, Aluminum potassium sulfate, Bordetella pertussis, Calcitriol, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), polyinosinic-polycytidylic acid stabilized with polylysine and carboxymethylcellulose (poly-ICLC, also known as Hiltonol), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, or Zymosan.

Some embodiments disclosed herein relate to methods for treating or preventing cancer, including administering a vaccine including a) one or more ncRNA peptides or b) a nucleic acid sequence encoding one or more ncRNA peptides. In some embodiments, the vaccine further includes an adjuvant. In some embodiments, the adjuvant is ABM2, AS01B, AS02, AS02A, Adjumer, Adjuvax, Algammulin, alum, aluminum phosphate, aluminum potassium sulfate, Bordetella pertussis, calcitriol, chitosan, cholera toxin, CpG, dibutyl phthalate, dimethyldioctadecylammonium bromide (DDA), Freund's adjuvant, Freund's complete, Freund's incomplete (IFA), GM-CSF, GMDP, gamma inulin, glycerol, HBSS (Hank's Balanced Salt Solution), polyinosinic-polycytidylic acid stabilized with polylysine and carboxymethylcellulose (poly-ICLC, also known as Hiltonol), IL-12, IL-2, imiquimod, interferon-gamma, ISCOM, lipid core peptide (LCP), lipofectin, lipopolysaccharide (LPS), liposomes, MF59, MLP+TDM, monophosphoryl lipid A, Montanide IMS-1313, Montanide ISA 206, Montanide ISA 720, Montanide ISA-51, Montanide ISA-50, nor-MDP, oil-in-water emulsion, P1005 (non-ionic copolymer), Pam3Cys (lipoprotein), pertussis toxin, poloxamer, QS21, RaLPS, Ribi, saponin, Seppic ISA 720, soybean oil, squalene, Syntex adjuvant formulation (SAF), synthetic polynucleotides (poly IC/poly AU), TiterMax tomatine, Vaxfectin, XtendIII, or Zymosan. In some embodiments, the vaccine is administered to a mammal. In some embodiments, the vaccine is administered to a human, a dog, a cat, a mouse, a rat, a rabbit, a horse, a cow, or a pig. In some embodiments, the cancer is selected from the group consisting of acute lymphoblastic leukemia, acute monocytic leukemia, acute myeloid leukemia, acute promyelocytic leukemia, adenocarcinoma, adult T-cell leukemia, astrocytoma, bladder cancer, bone cancer, brain tumor, breast cancer, Burkitt's lymphoma, carcinoma, cervical cancer, chronic lymphocytic leukemia, chronic myelogenous leukemia, colon cancer, colorectal cancer, endometrial cancer, glioblastoma multiforme, glioma, hepatocellular carcinoma, Hodgkin's lymphoma, inflammatory breast cancer, kidney cancer, leukemia, lung cancer, lymphoma, malignant mesothelioma, medulloblastoma, melanoma, multiple myeloma, neuroblastoma, non-Hodgkin lymphoma, non-small cell lung cancer, ovarian cancer, pancreatic cancer, pituitary tumor, prostate cancer, retinoblastoma, skin cancer, small cell lung cancer, squamous cell carcinoma, stomach cancer, T-cell leukemia, T-cell lymphoma, thyroid cancer, and Wilms' tumor. In some embodiments, treating the cancer includes reducing tumor size, inhibiting tumor growth, reducing tumor burden, increasing survival, or increasing cancer-free survival. In some embodiments, administering the vaccine elicits an immune response in the subject against the cancer.

Some embodiments disclosed herein relate to methods for treating a subject in need of treatment for a cancer. In some embodiments, the methods include a) obtaining a biological sample from the subject, b) identifying one or more ncRNA peptides that are immunoreactive with a biological sample from the subject in a first population of ncRNA peptides, c) preparing a vaccine composition including a second population of ncRNA peptides including one or more peptides identified in step b) or a nucleic acid sequence encoding the second population of peptides, and d) administering an effective amount of the vaccine composition to the subject, thereby treating the cancer. In some embodiments, identifying one or more ncRNA peptides that are immunoreactive includes contacting the biological sample with an array including a first population of ncRNA peptides, measuring binding of the biological sample to the first population of ncRNA peptides of the array, and determining a second population of ncRNA peptides of the array that are immunoreactive with the biological sample. In some embodiments, the second population of ncRNA peptides is a subpopulation of the first population of ncRNA peptides. In some embodiments, the methods further include determining a nucleic acid sequence of said ncRNA peptides to identify one or more nucleic acids encoding ncRNA peptides that are immunogenic, cancer-specific, and/or shared across cancers. In some embodiments, the biological sample is selected from the group consisting of blood, plasma, serum, thymus, bone marrow, spleen, lymph node, bronchoalveolar lavage, breast, central nervous system, cerebrospinal fluid, eye, tears, gastrointestinal tract, saliva, feces, urine, heart, kidney, liver, lung, muscle, pancreas, peripheral nervous system, saliva, skin, thyroid, trachea, and tumor. In some embodiments, the biological sample includes an antibody. In some embodiments, treating the cancer includes reducing tumor size, inhibiting tumor growth, reducing tumor burden, increasing survival, or increasing cancer-free survival. In some embodiments, administering the vaccine composition elicits an immune response in the subject against the cancer. In some embodiments, the vaccine composition further includes a pharmaceutically acceptable adjuvant or excipient. In some embodiments, the adjuvant is ABM2, AS01B, AS02, AS02A, Adjumer, Adjuvax, Algammulin, alum, aluminum phosphate, aluminum potassium sulfate, Bordetella pertussis, calcitriol, chitosan, cholera toxin, CpG, dibutyl phthalate, dimethyldioctadecylammonium bromide (DDA), Freund's adjuvant, Freund's complete, Freund's incomplete (IFA), GM-CSF, GMDP, gamma inulin, glycerol, HBSS (Hank's Balanced Salt Solution), polyinosinic-polycytidylic acid stabilized with polylysine and carboxymethylcellulose (poly-ICLC, also known as Hiltonol), IL-12, IL-2, imiquimod, interferon-gamma, ISCOM, lipid core peptide (LCP), lipofectin, lipopolysaccharide (LPS), liposomes, MF59, MLP+TDM, monophosphoryl lipid A, Montanide IMS-1313, Montanide ISA 206, Montanide ISA 720, Montanide ISA-51, Montanide ISA-50, nor-MDP, oil-in-water emulsion, P1005 (non-ionic copolymer), Pam3Cys (lipoprotein), pertussis toxin, poloxamer, QS21, RaLPS, Ribi, saponin, Seppic ISA 720, soybean oil, squalene, Syntex adjuvant formulation (SAF), synthetic polynucleotides (poly IC/poly AU), TiterMax tomatine, Vaxfectin, XtendIII, or Zymosan. In some embodiments, the cancer is selected from the group consisting of acute lymphoblastic leukemia, acute monocytic leukemia, acute myeloid leukemia, acute promyelocytic leukemia, adenocarcinoma, adult T-cell leukemia, astrocytoma, bladder cancer, bone cancer, brain tumor, breast cancer, Burkitt's lymphoma, carcinoma, cervical cancer, chronic lymphocytic leukemia, chronic myelogenous leukemia, colon cancer, colorectal cancer, endometrial cancer, glioblastoma multiforme, glioma, hepatocellular carcinoma, Hodgkin's lymphoma, inflammatory breast cancer, kidney cancer, leukemia, lung cancer, lymphoma, malignant mesothelioma, medulloblastoma, melanoma, multiple myeloma, neuroblastoma, non-Hodgkin lymphoma, non-small cell lung cancer, ovarian cancer, pancreatic cancer, pituitary tumor, prostate cancer, retinoblastoma, skin cancer, small cell lung cancer, squamous cell carcinoma, stomach cancer, T-cell leukemia, T-cell lymphoma, thyroid cancer, and Wilms' tumor. In some embodiments, the subject is a mammal. In some embodiments, the subject is a human, a dog, a cat, a mouse, a rat, a rabbit, a horse, a cow, or a pig.

Some embodiments provided herein relate to methods of treating or preventing cancer. In some embodiments, the methods include administering a therapeutic molecule designed to bind a ncRNAP. In some embodiments, the therapeutic molecule is an antibody or synthetic antibody. In some embodiments, the therapeutic molecule is administered in combination with another cancer treatment. In some embodiments, the cancer is selected from the group consisting of acute lymphoblastic leukemia, acute monocytic leukemia, acute myeloid leukemia, acute promyelocytic leukemia, adenocarcinoma, adult T-cell leukemia, astrocytoma, bladder cancer, bone cancer, brain tumor, breast cancer, Burkitt's lymphoma, carcinoma, cervical cancer, chronic lymphocytic leukemia, chronic myelogenous leukemia, colon cancer, colorectal cancer, endometrial cancer, glioblastoma multiforme, glioma, hepatocellular carcinoma, Hodgkin's lymphoma, inflammatory breast cancer, kidney cancer, leukemia, lung cancer, lymphoma, malignant mesothelioma, medulloblastoma, melanoma, multiple myeloma, neuroblastoma, non-Hodgkin lymphoma, non-small cell lung cancer, ovarian cancer, pancreatic cancer, pituitary tumor, prostate cancer, retinoblastoma, skin cancer, small cell lung cancer, squamous cell carcinoma, stomach cancer, T-cell leukemia, T-cell lymphoma, thyroid cancer, and Wilms' tumor. In some embodiments, the therapeutic molecule is administered to a mammal. In some embodiments, the therapeutic molecule is administered to a human, a dog, a cat, a mouse, a rat, a rabbit, a horse, a cow, or a pig. In some embodiments, treating the cancer includes reducing tumor size, inhibiting tumor growth, reducing tumor burden, increasing survival, or increasing cancer-free survival. In some embodiments, administering the therapeutic molecule elicits an immune response in the subject against the cancer.

Some embodiments provided herein relate to methods of producing a therapeutic or preventative antibody specific for cancer of interest. In some embodiments, the methods include contacting one or more biological samples obtained from one or more subjects identified as having the cancer of interest to an array including ncRNA peptides produced by one or more tumors; detecting antibodies which bind to the ncRNA peptides; selecting one or more ncRNA peptides that are immunoreactive with the antibodies; and preparing an antibody composition against the selected ncRNA peptides for the cancer of interest. In some embodiments, the ncRNA peptides of the array are spaced between 3 and 9 μM apart. In some embodiments, the array comprises at least one peptide having at least 95% sequence identity with any of SEQ ID NOs: 1-17. In some embodiments, the array includes about 10, about 100, about 200, about 300, about 500, about 1,000, about 2,000, about 3,000, about 5,000, about 7,500, about 10,000, about 12,500, about 15,000, about 17,500, about 20,000, about 22,500, about 25,000, about 27,500, about 30,000, about 32,500, about 35,000, about 37,500, about 40,000, about 50,000, about 100,000, about 200,000, about 300,000, about 400,000, about 500,000, about 600,000, about 700,000, about 800,000, about 900,000, about 1,000,000, about 1,500,000, about 2,000,000, about 2,500,000, about 3,000,000, about 3,500,000, about 4,000,000, about 4,500,000, or about 5,000,000 ncRNAPs. In some embodiments, the ncRNA peptides are in-situ synthesized on the array. In some embodiments, the ncRNA peptides are fixed on a substrate. In some embodiments, the substrate includes glass, silica, composite, resin, or combination thereof. In some embodiments, binding is detected by at least one of fluorescence, luminescence, calorimetry, chromatography, radioactivity, Bio-Layer Interferometry, electro-interference sensing, or surface plasmon resonance. In some embodiments, the biological sample is selected from the group consisting of blood, plasma, scrum, thymus, bone marrow, spleen, lymph node, bronchoalveolar lavage, breast, central nervous system, cerebrospinal fluid, eye, tears, gastrointestinal tract, saliva, feces, urine, heart, kidney, liver, lung, muscle, pancreas, peripheral nervous system, saliva, skin, thyroid, trachea, and tumor. In some embodiments, the cancer of interest is selected from the group consisting of acute lymphoblastic leukemia, acute monocytic leukemia, acute myeloid leukemia, acute promyelocytic leukemia, adenocarcinoma, adult T-cell leukemia, astrocytoma, bladder cancer, bone cancer, brain tumor, breast cancer, Burkitt's lymphoma, carcinoma, cervical cancer, chronic lymphocytic leukemia, chronic myelogenous leukemia, colon cancer, colorectal cancer, endometrial cancer, glioblastoma multiforme, glioma, hepatocellular carcinoma, Hodgkin's lymphoma, inflammatory breast cancer, kidney cancer, leukemia, lung cancer, lymphoma, malignant mesothelioma, medulloblastoma, melanoma, multiple myeloma, neuroblastoma, non-Hodgkin lymphoma, non-small cell lung cancer, ovarian cancer, pancreatic cancer, pituitary tumor, prostate cancer, retinoblastoma, skin cancer, small cell lung cancer, squamous cell carcinoma, stomach cancer, T-cell leukemia, T-cell lymphoma, thyroid cancer, and Wilms' tumor.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments, and are not intended to be limiting in scope.

FIGS. 1A-1D illustrate exemplary roles of ncRNA-encoded peptides in cancer. FIG. 1A demonstrates that the lncRNA HOXB-AS3 encodes a small peptide that suppresses PKM slicing and subsequence metabolic reprogramming by competitively binding to hnRNPA1. FIG. 1B demonstrates that the lncRNA LOC90024 produces a small oncoprotein (SRSP). SRSP interacts with SRSF3 to increase the binding of SRSF3 to exon 3 of the transcription factor Sp4, which results in the formation of the “cancerous” long Sp4 isoform (L-Sp4 protein). FIG. 1C demonstrates that LINC00266-1 encodes a 71-aa peptide (RBRP). RBRP interacts with the m6A reader IGF2BP1, which results in enhanced m6A recognition by IGF2BP1 on the mRNA of the oncogene c-Myc to increase the stability of the mRNA and the level of c-Myc by strengthening the recruitment of the RNA stabilizers HuR, MATK3, and PABPC1. FIG. 1D demonstrates that LINC00998 encodes a conserved 59 aa peptide, SMIM30. SMIM30 is an important adaptor for the membrane anchoring and activation state of the tyrosine kinases, SRC/YES1, resulting in activating the mitogen-activated protein kinase (MAPK) signaling pathway to promote HCC development.

### DETAILED DESCRIPTION

In the following detailed description, reference is made to the accompanying drawings which form a part hereof, and in which are shown by way of illustration embodiments that may be practiced. It is to be understood that other embodiments may be utilized, and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A) B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Unless otherwise noted, technical terms are used according to conventional usage. Definitions of common terms in molecular biology can be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: A Comprehensive Desk Reference, published by VCH Publishers, Inc., 1995 (ISBN 9780471185710); and other similar references. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

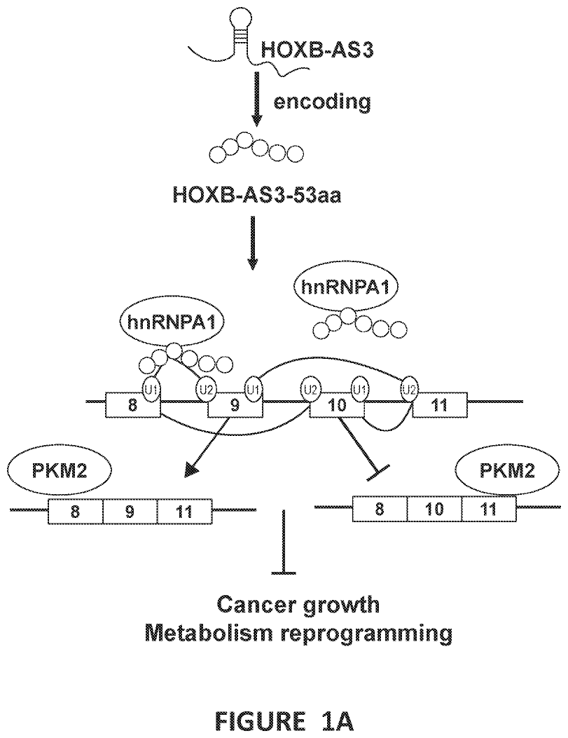

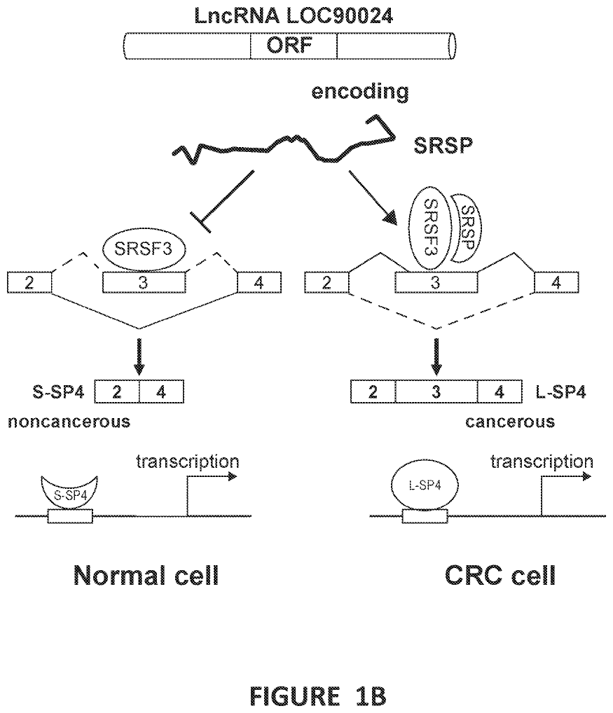

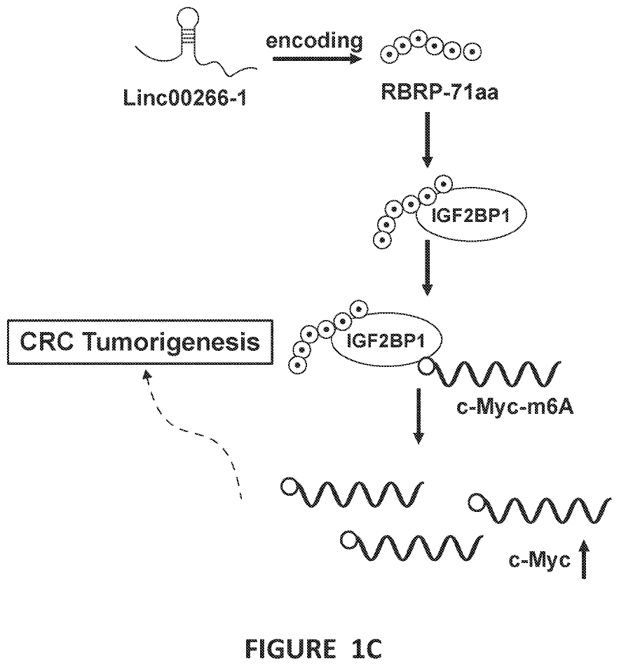

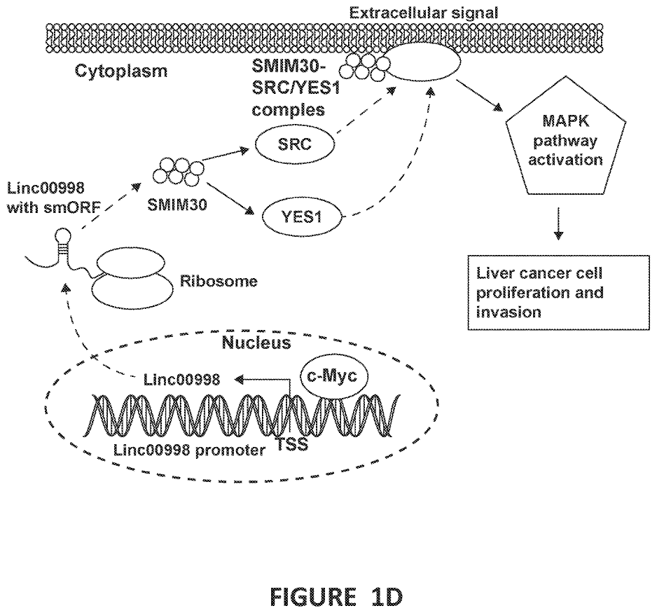

Provided herein are methods and compositions for preventing, treating, and diagnosing cancer including the use of neoantigens. Neoantigens herein include peptides encoded by nucleic acids in a tumor that are recognized as foreign by the subject's immune system. As described here, these neoantigens are encoded by various forms of non-coding RNA (ncRNA) in tumor versus normal cells (FIGS. 1A-1D). Also described are arrays that make it feasible to discover ncRNA peptides (ncRNAPs) for use in therapeutic or preventative cancer vaccines, in diagnostics for the detection of cancer in a subject or predicting response to immunotherapy, and as therapeutics for treatment of cancer.

The advent of immune-therapeutics, particularly checkpoint inhibitors, has revolutionized cancer treatment. However, only a small fraction of patients responds to therapy, it has risks of adverse events and is expensive. The success of checkpoint inhibitors in cancer therapy is largely attributed to activating the patient's immune response to their tumor's neoantigens arising from DNA mutations. This realization has motivated the interest in personal cancer vaccines, and possibly in shared antigen vaccines, based on sequencing the patient's tumor DNA to discover neoantigens in the hopes of expanding the benefit to patients beyond that of checkpoint inhibitors alone.

However, given that most DNA encoded neoantigens are personal and only a small percent is immunogenic, another source of neoantigens was investigated, and a new source was discovered in the RNA of a tumor. Tumors are very error-prone, relative to normal cells, in making and processing RNA. This includes mis-transcription through microsatellites, mis-initiation of translation of exon 1, mis-splicing of exons by excision or retention and mistranslation at tryptophan. All these processes create neoantigen peptides that are specific to tumors, shared between tumors, and highly immunogenic. These RNA Error Derived Neoantigens (REDN) peptides can be used in arrays for diagnosis of cancer, prediction of immune therapy response, as a source of therapeutic antibodies and vaccines.

Some embodiments provided herein relate to ncRNAs, which have potential as a rich source of cancer neoantigens. Also provided herein are methods of discovering such antigens.

Only about 2% of the genome actually encodes canonical genes. For many years, the 98% non-coding DNA was thought to be junk. However, it was discovered that much of the 98% “junk” is actually transcribed by the RNA polymerase. Since transcribed regions are much more likely to be repaired, the transcription of “junk” DNA may be a mode of preserving DNA integrity. The RNA polymerase would occasionally translate this RNA to peptides or proteins and this process was more likely to occur in tumors than normal cells (see WO 2009/126718 A2, hereby incorporated by reference in its entirety). It was later discovered that these non-coding RNAs often had biological functions as RNA. More recently, due to advances in RNA sequencing and ribosome profiling, it is becoming increasingly clear these ncRNAs do indeed encode peptides and small proteins, and these also often have biological functions. Some of the ncRNAs and the ncRNAPs have cancer specific expression.

Different forms of ncRNAs exist, as shown in FIGS. 1A-1D. The largest class is long, non-coding RNAs (lncRNA). There are also micro RNAs (miRNA), circular RNAs (circRNA), and RNA from pseudogenes. These ncRNAs are translated by the ribosome, often using non-canonical start and stop signals. A number of informatic and sequencing approaches have been developed to predict what parts of the ncRNAs may encode peptides or proteins (reviewed in Zhao et al., Translation of noncoding RNAs and cancer, Cancer Letters 497 (2021) 89-99, hereby incorporated by reference in its entirety).

It is now recognized that the ncRNA may enable diagnosis and therapy for cancer. However, to date it has only been proposed to use the ncRNA itself as a biomarker or therapeutic target, or biologically active peptides or proteins encoded by ncRNA as biomarkers or therapeutic drug targets. One company, EnaraBio has proposed that “Dark Antigens” presented on tumor cells may be a source of neoantigens for vaccines. EnaraBio uses mass spectrometry to identify such antigens. As these peptides are expressed at exceptionally low levels and given that mass spectrometry is a technically demanding approach to apply to large number of tumor samples, it would be very difficult, if at all possible, to find shared ncRNAPs that could be a source of diagnostics and vaccines.

Provided herein and systems and methods overcoming the challenges associated with mass spectrophotometric approaches. In some embodiments, ncRNAPs that are made and that arc immunogenic across many individuals are discovered using the systems and methods provided herein. In some embodiments, the systems and methods relate to a process for in-situ synthesis of up to five million peptides/slide using mask-based photolithography in an Intel-like process. This affords a method to screen the potentially millions of ncRNAPs for those that are immunogenic, tumor specific and shared. These systems and methods represent the only feasible approach for plumbing the ncRNAP space for peptides useful for cancer diagnostics, vaccines, and therapeutics.

FIGS. 1A-1D illustrate roles of lncRNA-encoded peptides in cancer. In FIG. 1A, the lncRNA HOXB-AS3 encodes a small peptide that suppresses PKM splicing and subsequent metabolic reprogramming by competitively binding to hnRNPA1. In FIG. 1B, the lncRNA LOC90024 produces a small oncoprotein (SRSP). SRSP interacts with SRSF3 to increase the binding of SRSF3 to exon 3 of the transcription factor Sp4, which results in the formation of the “cancerous” long Sp4 isoform (L-Sp4 protein). In FIG. 1C, LINC00266-1 encodes a 71-aa peptide (RBRP). RBRP interacts with the m6A reader IGF2BP1, which results in enhanced m6A recognition by IGF2BP1 on the mRNA of the oncogene c-Myc to increase the stability of the mRNA and the level of c-Myc by strengthening the recruitment of the RNA stabilizers HuR, MATK3, and PABPC1. In FIG. 1D, LINC00998 encodes a conserved 59 aa peptide, SMIM30. SMIM30 is an important adaptor for the membrane anchoring and activation state of the tyrosine kinases, SRC/YES1, resulting in activating the mitogen-activated protein kinase (MAPK) signaling pathway to promote HCC development.

Vaccines containing ncRNAPs (or nucleic acids encoding ncRNAPs) and therapeutic molecules that bind to ncRNAPs may be administered to an individual in need thereof in combination with other cancer therapeutics, including immunotherapy and RNA Error Derived Neoantigens (REDNs)-based therapy.

As used herein, the term “ncRNA” refers to non-coding RNAs, such as RNA transcripts which are transcribed from the portion of the genome not traditionally thought to encode a gene (so-called “junk DNA”) and/or from the dark proteome. ncRNAs have been thought to lack open reading frames (ORFs), nevertheless ncRNAs may be translated in a noncanonical mode. As used herein, “ncRNA” may include long noncoding RNAs (lncRNAs), microRNAs (miRNAs), circular RNAs (circRNAs), and RNA from pseudogenes, among other types. Like mRNAs, ncRNAs and pri-miRNAs with small ORFs may be characterized by 5′ capping and 3′ polyadenylation. In addition, circRNAs contain internal ribosome entry sites (IRESs) and N6-methyladenosine (m6A) modifications that might initiate translation, and the majority of circRNAs spliced from coding genes contain ORFs.

As used herein, the terms “ncRNA peptide” or “ncRNAP” refer to peptides encoded by an ncRNA. ncRNAPs may be produced when an ncRNA is translated by a ribosome. ncRNA peptides may also be produced synthetically, for example on a peptide array. An ncRNA peptide may be predicted from the “dark proteome,” which refers to proteins with no defined three-dimensional structure.

As used herein, the term “array” refers to an arrangement of molecules, such as biological macromolecules (such as peptides), in addressable locations on or in a substrate. A “microarray” is an array that is miniaturized so as to require or be aided by microscopic examination for evaluation or analysis. The array of molecules (“features”) makes it possible to carry out a very large number of analyses on a sample at one time. Within an array, each arrayed sample is addressable, in that its location can be reliably and consistently determined within at least two dimensions of the array. The feature application location on an array can assume different shapes. For example, the array can be regular (such as arranged in uniform rows and columns) or irregular. Thus, in ordered arrays the location of each sample is assigned to the sample at the time when it is applied to the array, and a key may be provided in order to correlate each location with the appropriate target or feature position. Often, ordered arrays are arranged in a symmetrical grid pattern, but samples can be arranged in other patterns (such as in radially distributed lines, spiral lines, or ordered clusters). Addressable arrays usually are computer readable, in that a computer can be programmed to correlate a particular address on the array with information about the sample at that position (such as hybridization or binding data, including for instance signal intensity). In some examples of computer readable formats, the subject features in the array are arranged regularly, for instance in a Cartesian grid pattern, which can be correlated to address information by a computer. In some cases, the methods provided herein involve multiplexed arrays in which a plurality of peptides or polypeptides attached to a solid support are contacted to a biological sample (e.g., blood or other bodily tissue obtained from a subject).

In certain embodiments, the peptide array is a plurality of short linear peptides immobilized on a solid surface (e.g., a polystyrene or other solid substrate). As used herein, the terms “peptide” and “polypeptide” refer to a polymer in which the monomers are alpha amino acids joined together through amide bonds. Peptides are two or often more amino acid monomers long. Standard abbreviations for amino acids are used herein (see Stryer, 1988, Biochemistry, Third Ed., incorporated herein by reference). In certain embodiments, random-sequence peptide arrays are used. As used herein, the term “substrate” refers to any type of solid support to which the peptides are immobilized. Examples of substrates include, but are not limited to, microarrays; beads; columns; optical fibers; wipes; nitrocellulose; nylon; glass; quartz; diazotized membranes (paper or nylon); silicones; polyformaldehyde; cellulose; cellulose acetate; paper; ceramics; metals; metalloids; semiconductive materials; coated beads; magnetic particles; plastics such as polyethylene, polypropylene, and polystyrene; gel-forming materials; silicates; agarose; polyacrylamides; methylmethracrylate polymers; sol gels; porous polymer hydrogels; nanostructured surfaces; nanotubes (such as carbon nanotubes); and nanoparticles (such as gold nanoparticles or quantum dots). When bound to a substrate, the peptides can be directly linked to the support, or attached to the surface via a linker. Thus, the solid substrate and/or the peptides can be derivatized using methods known in the art to facilitate binding of the peptides to the solid support, so long as the derivatization does not eliminate detection of binding between the peptides and antibodies in the sera.

In certain embodiments, the ncRNA peptide array includes a number of ncRNAPs ranging from about 10 to about 5 million. In some embodiments, the ncRNA peptide array includes at least about 10, about 100, about 200, about 300, about 500, about 1,000, about 2,000, about 3,000, about 5,000, about 7,500, about 10,000, about 12,500, about 15,000, about 17,500, about 20,000, about 22,500, about 25,000, about 27,500, about 30,000, about 32,500, about 35,000, about 37,500, about 40,000, about 50,000, about 100,000, about 200,000, about 300,000, about 400,000, about 500,000, about 600,000, about 700,000, about 800,000, about 900,000, about 1,000,000, about 1,500,000, about 2,000,000, about 2,500,000, about 3,000,000, about 3,500,000, about 4,000,000, about 4,500,000, or about 5,000,000 ncRNAPs, or a number of ncRNAPs within a range defined by any two of the aforementioned values.

As used herein, “in-situ synthesis” refers to synthesis of peptides or polypeptides in situ on an array. This could be done with photoactivatable amino acids as done by Nimble Therapeutics (maskless photolithography), PEPperPRINT, a standard mask-based system (much like Intel uses to lay down circuits), BOC or FMOC peptide chemistry, or other synthesis methods known in the art.

As used herein, the term “detect,” “detection,” “detectable,” or “detecting” is understood both on a quantitative and a qualitative level, as well as a combination thereof. It thus includes quantitative, semi-quantitative, and qualitative measurements of measuring a cancer in a subject, or measuring binding of a biological sample to ncRNA peptides, using the methods and compositions as disclosed herein.

As used herein, the term “cancer-specific” or “tumor-specific” refers to ncRNA peptides that are found in a particular cancer and/or tumor type. “Cancer-specific” or “tumor-specific” also refers to ncRNA peptides that are reactive with two or more biological samples from individuals with a particular cancer or tumor-type. Cancer-specific or tumor-specific ncRNAPs may be determined by observing binding between two or more biological samples from individuals with a particular cancer or tumor type and an array of ncRNAPs and identifying one or more ncRNAPs that are bound by two or more of the samples. Samples from control subjects without cancer may also be compared. Such cancer-specific ncRNAPs may be found in multiple particular cancers but not generally across cancers.

As used herein, the term “shared across cancers” refers to ncRNA peptides that are found in several cancer and/or tumor types. “Shared across cancers” also refers to ncRNA peptides that are reactive with two or more biological samples from individuals with a different cancer or tumor-types. Shared ncRNAPs may be determined by observing binding between two or more biological samples from individuals with different cancer or tumor types and an array of ncRNAPs and identifying one or more ncRNAPs that are bound by two or more of the samples. Samples from control subjects without cancer may also be compared to reduce false positives.

As used herein, the term “mammal” includes both human and non-human mammals. Similarly, the term “subject” includes both human and veterinary subjects, including dogs.

As used herein, the expression “a subject in need thereof” or “individual in need thereof” means a human or non-human mammal that exhibits one or more symptoms or indications of cancer, and/or who has been diagnosed with cancer, including a solid tumor and who needs treatment for the same. In many embodiments, the term “subject” may be interchangeably used with the term “patient.” For example, a human subject may be diagnosed with a primary or a metastatic tumor and/or with one or more symptoms or indications including, but not limited to, unexplained weight loss, general weakness, persistent fatigue, loss of appetite, fever, night sweats, bone pain, shortness of breath, swollen abdomen, chest pain/pressure, enlargement of spleen, and elevation in the level of a cancer-related biomarker.

The term “malignancy” refers to a non-benign tumor or a cancer. As used herein, the term “cancer” includes a malignancy characterized by deregulated or uncontrolled cell growth. Exemplary cancers include carcinomas, sarcomas, leukemias, and lymphomas. Cancer includes primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor). A cancer may include, for example, gastric, myeloid, colon, nasopharyngeal, esophageal, and prostate tumors, glioma, neuroblastoma, breast cancer, lung cancer, ovarian cancer, colorectal cancer, thyroid cancer, leukemia (e.g., adult T-cell leukemia, acute monocytic leukemia, acute myeloid leukemia, acute promyelocytic leukemia, myelogenous leukemia, lymphocytic leukemia, acute myelogenous leukemia (AML), chronic myeloid leukemia (CML), acute lymphoblastic leukemia (ALL), T-lineage acute lymphoblastic leukemia or T-ALL chronic lymphocytic leukemia (CLL), myelodysplastic syndrome (MDS), hairy cell leukemia), lymphoma (Hodgkin's lymphoma (HL), non-Hodgkin's lymphoma (NHL)), multiple myeloma, bladder, renal, gastric (e.g., gastrointestinal stromal tumors (GIST)), liver, melanoma and pancreatic cancer, sarcoma, adenocarcinoma, astrocytoma, Bone cancer, brain tumor, Burkitt's lymphoma, carcinoma, cervical cancer, chronic lymphocytic leukemia, chronic myelogenous leukemia, endometrial cancer, glioblastoma multiforme, glioma, hepatocellular carcinoma, Hodgkin's lymphoma, inflammatory breast cancer, kidney cancer, leukemia, lymphoma, Hodgkin lymphoma, non-small cell lung cancer, pancreatic cancer, pituitary tumor, retinoblastoma, skin cancer, small cell lung cancer, squamous cell carcinoma, stomach cancer, T-cell leukemia, T-cell lymphoma, and Wilms' tumor.

As used herein, the term “pooled” refers to a plurality of components that have been combined to create a new composition. For example, a vaccine composition may comprise two or more ncRNA peptides.

As used herein, the term “sample” means non-biological samples and biological samples. Non-biological samples include those prepared in vitro including varying concentrations of a target molecule of interest in solution. Biological samples include, without limitation, blood, lymph, urine, saliva, sputum, other bodily secretions, cells, and tissue specimens and dilutions of them. Any suitable biological sample can be used. For example, a biological sample can be a specimen obtained from a subject {e.g., a mammal such as a human, canine, mouse, rat, pig, guinea pig, cow, monkey, or ape) or can be derived from such a subject. A subject can provide a plurality of biological sample, including a solid biological sample, from for example, a biopsy or a tissue. In some cases, a sample can be a tissue section or cells that are placed in or adapted to tissue culture. A biological sample also can be a biological fluid such as urine, blood, plasma, serum, saliva, tears, or mucus, or such a sample absorbed onto a paper or polymer substrate. A biological sample can be further fractionated, if desired, to a fraction containing particular cell types. In some embodiments, a sample can be a combination of samples from a subject (e.g., a combination of a tissue and fluid sample). In some cases, sera are obtained from the individual using techniques known in the art. In some embodiments, a subject can, for example, use a “fingerstick,” or “fingerprick” to draw a small quantity of blood and add it to a surface, such as a filter paper or other absorbent source, or in a vial or container and optionally dried. A biological sample obtained, for example, from a drop of a subject's blood and placed on a filter paper can be directly mailed to a provider of the methods of the invention without a processing of the sample. A biological sample provided by a subject can be concentrated or diluted.

As used herein, the term “contacting” includes placement in direct physical association, including solid or liquid forms. As used herein, “binding” refers to an association between two substances or molecules, such as the association of an antibody with a peptide. Binding can be detected by any procedure known to one skilled in the art, such as by physical or functional properties of the formed complexes, such as a target/antibody complex.

As used herein, the term “control” means a sample or standard used for comparison with an experimental sample, such as a tumor sample obtained from a patient with a particular type of cancer. The control can be a sample obtained from a healthy patient or a non-tumor tissue sample obtained from a patient diagnosed with a particular type of cancer. A control can also be a historical control or standard reference value or range of values (such as a previously tested control sample, such as a group of cancer patients with poor prognosis, or group of samples that represent baseline or normal values). A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

The methods provided herein are sensitive and involve small quantities of biological samples from a subject. In some embodiments, biological samples from a subject are too concentrated and require a dilution prior to being contacted with an array of the invention. A plurality of dilutions can be applied to a biological sample prior to contacting the sample with an array of the invention. A dilution can be a serial dilution, which can result in a geometric progression of the concentration in a logarithmic fashion. For example, a ten-fold serial dilution can be 1 M, 0.01 M, 0.001 M, and a geometric progression thereof. A dilution can be, for example, a one-fold dilution, a two-fold dilution, a three-fold dilution, a four-fold dilution, a five-fold dilution, a six-fold dilution, a seven-fold dilution, an eight-fold dilution, a nine-fold dilution, a ten-fold dilution, a sixteen-fold dilution, a twenty-five-fold dilution, a thirty-two-fold dilution, a sixty-four-fold dilution, and/or a one-hundred-and-twenty-five-fold dilution.

The binding of a molecule to an array in accordance with certain embodiments of the methodology disclosed herein creates a pattern of binding that can be associated with a condition. The affinity of binding of a molecule to a peptide in the array can be mathematically associated with a condition. The off-target binding pattern of an antibody to a plurality of different peptides of the invention can be mathematically associated with a condition. The avidity of binding of a molecule to a plurality of different peptides can be mathematically associated with a condition.

The peptide array can be contacted with the biological sample (e.g., sera) under any suitable conditions to promote binding of antibodies in the sample to peptides immobilized on the array. Thus, the methods presented herein are not limited by any specific type of binding conditions employed. Such conditions will vary depending on the array being used, the type of substrate, the density of the peptides arrayed on the substrate, desired stringency of the binding interaction, and nature of the competing materials in the binding solution. In certain embodiments, the conditions include a step to remove unbound antibodies from the addressable array.

Similarly, any suitable detection technique can be used in the methods provided herein to detect binding of antibodies (“antibody reactivity”) in the biological sample to peptides on the array. Such reactivity may be measured or estimated in any operable way, such as, for example, by ELISA or by microarray assay. In one embodiment, any type of detectable label can be used to label peptides on the array, including but not limited to radioisotope labels, fluorescent labels, luminescent labels, and electrochemical labels (for example, ligand labels with different electrode mid-point potential, where detection includes detecting electric potential of the label). Alternatively, bound antibodies can be detected, for example, using a detectably labeled secondary antibody.

In certain embodiments, the subject has been diagnosed with cancer or other cell proliferative disorder. As used herein, the term “cancer” refers to the broad class of disorders characterized by hyperproliferative cell growth, either in vitro (e.g., transformed cells) or in vivo. Cancers appropriate for treatment with checkpoint inhibitor therapy include without limitation a variety of neoplasms, including benign or malignant tumors, a variety of hyperplasias, and the like. Non-limiting examples of cancers that can be diagnosed, monitored, prevented, and/or treated with an array and a method of the invention can include: acute lymphoblastic leukemia, acute myeloid leukemia, adrenocortical carcinoma, AIDS-related cancers, AIDS-related lymphoma, anal cancer, appendix cancer, astrocytomas, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma Merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenstrom macroglobulinemia, and Wilms tumor.

Optionally, it is useful to determine immunogenicity of a candidate ncRNA peptide for use in a cancer vaccine. Immunogenicity, as used herein, refers to the ability of a substance, such as a peptide, to elicit an immune response, such as an antibody response or a T cell response, when administered to an individual, for example, in a vaccine formulation. As used herein, “immune response” means a response of a cell of the immune system, such as a B cell, T cell, or monocyte, to a stimulus. For individuals with cancer, it is the immune response to the tumor. In some embodiments, a peptide that reacts with an antibody or elicits T cell activity in a biological sample from an individual is not immunogenic when administered in a vaccine formulation. In some embodiments, a peptide that reacts with an antibody or elicits T cell activity in a biological sample from an individual is immunogenic when administered in a vaccine formulation. Immunogenicity is determined by methods of those of skill in the art including in animal model testing and using in silico prediction of immunogenicity. In silico immunogenicity prediction tools are available for free to the public, for example at the Immune Epitope Database and Analysis Resource (www.iedb.org). As used herein, an immunogenic peptide is a peptide which includes an allele-specific motif or other sequence such that the peptide will bind an MHC molecule and induce a cytotoxic T lymphocyte (“CTL”) response, or a B cell response (e.g., antibody production) against the antigen from which the immunogenic peptide is derived.

Immunogenic peptides include synthetic embodiments of peptides described herein. In addition, analogs (non-peptide organic molecules), derivatives (chemically functionalized peptide molecules obtained starting with the disclosed peptide sequences) and variants (homologs) of these proteins can be utilized in the methods described herein. Each polypeptide of this disclosure is made of a sequence of amino acids, which may be either L- and/or D-amino acids, naturally occurring and otherwise.

Peptides can be modified by a variety of chemical techniques to produce derivatives having essentially the same activity as the unmodified peptides, and optionally having other desirable properties. For example, carboxylic acid groups of the protein, whether carboxyl-terminal or side chain, can be provided in the form of a salt of a pharmaceutically acceptable cation or esterified to form a C1-C16 ester, or converted to an amide of formula NR1R2 wherein R1 and R2 are each independently H or C1-C16 alkyl, or combined to form a heterocyclic ring, such as a 5- or 6-membered ring. Amino groups of the peptide, whether amino-terminal or side chain, can be in the form of a pharmaceutically acceptable acid addition salt, such as the HCl, HBr, acetic, benzoic, toluene sulfonic, maleic, tartaric and other organic salts, or can be modified to C1-C16 alkyl or dialkyl amino or further converted to an amide.

Hydroxyl groups of the peptide side chains may be converted to C1-C16 alkoxy or to a C1-C16 ester using well-recognized techniques. Phenyl and phenolic rings of the peptide side chains may be substituted with one or more halogen atoms, such as fluorine, chlorine, bromine, or iodine, or with C1-C16 alkyl, C1-C16 alkoxy, carboxylic acids and esters thereof, or amides of such carboxylic acids. Methylene groups of the peptide side chains can be extended to homologous C2-C4 alkylenes. Thiols can be protected with any one of a number of well-recognized protecting groups, such as acetamide groups. Those skilled in the art will also recognize methods for introducing cyclic structures into the peptides of this disclosure to select and provide conformational constraints to the structure that result in enhanced stability.

Peptidomimetic and organomimetic embodiments are envisioned, whereby the three-dimensional arrangement of the chemical constituents of such peptido- and organomimetics mimic the three-dimensional arrangement of the peptide backbone and component amino acid side chains, resulting in such peptido- and organomimetics of an immunogenic Brachyury polypeptide having measurable or enhanced ability to generate an immune response. For computer modeling applications, a pharmacophore is an idealized three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs,” in Klegerman & Groves, eds., 1993, Pharmaceutical Biotechnology, Interpharm Press: Buffalo Grove, Ill., pp. 165-174 and Principles of Pharmacology, Munson (ed.) 1995, Ch. 102, for descriptions of techniques used in CADD. Also included are mimetics prepared using such techniques.

As used herein, the term “diagnose” or “diagnostic” means identifying the presence or nature of a pathologic condition. Diagnostic methods differ in their sensitivity and specificity. The “sensitivity” of a diagnostic assay is the percentage of diseased subjects who test positive (percent of true positives). The “specificity” of a diagnostic assay is 1 minus the false positive rate, where the false positive rate is defined as the proportion of those without the disease who test positive. While a particular diagnostic method may not provide a definitive diagnosis of a condition, it suffices if the method provides a positive indication that aids in diagnosis. “Prognostic” means predicting the probability of development (for example, severity) of a pathologic condition.

As used herein, the term “treat” or “treatment” refers to a method of reducing the effects of a disease or condition. Treatment can also refer to a method of reducing the disease or condition itself rather than just the symptoms. The treatment can be any reduction from native levels and can be but is not limited to the complete ablation of the disease, condition, or the symptoms of the disease or condition. For example, a disclosed method for reducing the effects of a cancer is considered to be a treatment if there is a 10% reduction in one or more symptoms of the disease (e.g., tumor size) in a subject with the disease when compared to native levels in the same subject or control subjects. Thus, the reduction can be a 10, 20, 30, 40, 50, 60, 70, 80, 90, 100%, or any amount of reduction in between as compared to native or control levels. It is also understood and contemplated herein that treatment can refer to any reduction in the progression of a disease or cancer. Thus, for example, methods of reducing the effects of a cancer are considered to be a treatment if there is a 10% reduction in the tumor growth rate relative to a control subject or tumor growth rates in the same subject prior to the treatment. It is understood that the reduction can be a 10, 20, 30, 40, 50, 60, 70, 80, 90, 100%, or any amount of reduction in between as compared to native or control levels.

As used herein the term “immunotherapeutic,” “immunotherapy,” or “IT” refers to a compound that is used to, in this case, treat cancer by inducing, enhancing, or suppressing the immune response. Immunotherapeutics encompass immune checkpoint inhibitors, antibody-drug conjugates (ADCs), monoclonal antibodies, T-cell therapy, small molecules, and bispecific antibodies (bsAbs). Antibody-drug conjugates include monoclonal antibodies linked to biologically active drugs to combine the targeting ability of antibodies as well as the cytotoxic ability of the drug. T-cell therapy involves reprogramming a patient's own immune T cells to attack tumors. One type of well-known T-cell therapy includes adoptive transfer of chimeric antigen receptor (CAR) T-cells. As used herein, the term “chimeric antigen receptor” refers to a fusion protein of the membrane or intracellular signaling region of T-cell activating proteins (e.g., CD3-zeta chain, CD28, 41BBL, OX40, ICOS, high-affinity receptor for IgE (FcsRI) and other T-cell activating proteins) and the antigen-binding site (for example, a single-chain Fv fragment) of a cancer antigen-specific antibody. Bispecific antibodies are recombinant proteins that can bind to two different types of antigens at the same time. For example, a bsAb can be engineered to bind a cytotoxic cell and a target tumor cell. That way, the bsAb brings the cytotoxic cell and the target tumor cell into close proximity and facilitates tumor treatment.

Immune checkpoint inhibitor therapy (CPI or ICI) is a form of cancer immunotherapy. The therapy targets immune checkpoints, key regulators of the immune system that when stimulated can dampen the immune response to an immunologic stimulus. Some cancers can protect themselves from attack by stimulating immune checkpoint targets. Checkpoint therapy can block inhibitory checkpoints, restoring immune system function. Currently approved checkpoint inhibitors target the molecules CTLA4, PD-1, and PD-L1. PD-1 is the transmembrane programmed cell death 1 protein (also called PDCD1 and CD279), which interacts with PD-L1 (PD-1 ligand 1, or CD274). PD-L1 on the cell surface binds to PD1 on an immune cell surface, which inhibits immune cell activity. Among PD-L1 functions is a key regulatory role on T cell activities. It appears that (cancer mediated) upregulation of PD-L1 on the cell surface may inhibit T cells that might otherwise attack. Antibodies that bind to either PD-1 or PD-L1 and therefore block the interaction may allow the T cells to attack the tumor.

Immune checkpoint inhibitors, such as anti-PD-1 antibodies, have been approved to treat different types of cancer (e.g., bladder, lung, kidney, melanoma, head, neck, Hodgkin's lymphoma, and solid tumors). PD-1 inhibitors include nivolumab, pembrolizumab, cemiplimab and spartalizumab. Additional CPIs include CTLA-4 blockage (e.g., ipilimumab, such as for treatment of melanoma) and PD-LI inhibitors (e.g., atezolizumab, avelumab, or durvalumab, such as for treatment of bladder cancer). Remarkably, the FDA for the first time gave tumor-type, agnostic approval to treat any late-stage cancer that is MSI-H. This was based on the remarkably positive responses to treatment of not only cancers with frequent MSI-H phenotypes (colon, endometrial and stomach), but rare MSI-H patients in other cancers. For example, a woman with triple negative, metastatic breast cancer who was MSI-H had a complete remission, while most breast cancers have been unresponsive to CPI treatment.

Examples of immunotherapeutic include Tremelimumab (CTLA-4 blocking antibody), OX40 agonists (e.g., agonist antibodies), antibodies to B7 ligands (e.g., anti-B7-H1, anti-B7-H3, anti-B7-H3, anti-B7-H4), durvalumab (MEDI4736, anti-PD-L1 antibody), MK-3475 (PD-1 blocker), Nivolumab (anti-PD-1 antibody), Pembrolizumab (anti-PD-1 antibody), Pidilizumab/CT-Oi 1, BY55 monoclonal antibody, AMP224 (anti-PD-L1 antibody), BMS-936559 (anti-PD-L1 antibody), MPLDL3280A (anti-PD-L1 antibody), MSB0010718C (anti-PD-L1 antibody), and Yervoy/ipilimumab (anti-CTLA-4 checkpoint inhibitor). Many new inhibitor targets are being investigated. In some cases, IT treatment includes a combination therapy in which two or more immunotherapeutics are administered.

As used herein the terms “checkpoint inhibitor” and “checkpoint pathway inhibitor” are used interchangeably and refer to negative regulatory molecules, usually antibodies, that block or inhibit anti-T cell anti-tumor function to enhance tumor killing. Checkpoint inhibitors include, without limitation, CTLA-4, PD-L1, PD-L2, PD-1, B7-H3, B7-H4, BTLA, HVEM, TEVI3, GAL9, LAG3, VISTA, KIR, 2B4, CD160, CGEN-15049, CHK 1, CHK2, A2aR, and a B-7 family ligand such as B7-1, B7-2, B7-DC, B7-H1, B7-H2, B7-H3, B7-H4, B7-H5, B7-H6 and B7-H7 (or any combination thereof), or a combination thereof (e.g., a combination of CTLA-4 and PD-L1 or PD-L2).

As used herein, the term “adverse event, “adverse reaction,” “adverse response,” “adverse immune response,” “side effect” or “side effects” refers to the unacceptable or undesirable adverse symptoms resulting from or associated with the administration of a particular treatment such as an IT therapy. Side effects specifically to immunotherapeutics are termed “immune related adverse events” (irAE). While side effects vary by the type of therapy, common side effects of IT therapies include, without limitation fatigue, infusion related reactions, dermatological toxicity, diarrhea/colitis, hepatotoxicity, pneumonitis, hyper- and hypothyroidism. For review, see e.g., uptodate.com/contents/patient-selection-criteria-and-toxicities-associated-with-checkpoint-inhibitor-immunotherapy on the World Wide Web. Immune-related adverse events are generally graded from 1-4. Grades 3 and 4 are considered serious and can require immunosuppression treatment. Patients with irAE are just as likely to have a positive response to treatment. Occurrence of Grade 3 or 4 event can prohibit the patient from further IT therapy. Therefore, knowing ahead of time which patients are more likely to have an event would allow closer monitoring to preempt a Grade 3 or 4 event.

Any appropriate criteria can be used to confirm a subject's responsiveness to treatment with an IT. For example, in certain embodiments, responsiveness to treatment by an IT is measured by at least one criterion selected from the group consisting of clinical benefit rate, survival until mortality, pathological complete response, semi-quantitative measures of pathologic response, clinical complete remission, clinical partial remission, clinical stable disease, recurrence-free survival, metastasis free survival, disease free survival, circulating tumor cell decrease, circulating marker response, and RECIST criteria.

The methods described herein can be carried out using a computer programmed to receive data (e.g., data from a ncRNA array indicating whether a subject has a binding signature associated with cancer, associated with responsiveness to IT therapy, or associated with adverse reactions to IT therapy). The computer can output for display information related to a subject's biomarkers, and the likelihood of the duration of time that the subject will be responsive to an IT therapy, suffer a side-effect, or the prognosis of survival.

After information regarding a subject's biomarkers is reported, a professional can take one or more actions that can affect patient care (e.g., administer a new treatment or modify an existing treatment). For example, a medical professional can record the information in a subject's medical record and/or in an electronic database. In some cases, a medical professional can record that the subject is likely or not likely to respond to an IT therapy, or otherwise transform the patient's medical record, to reflect the patient's medical condition. In some cases, a medical professional can review and evaluate a patient's medical record, and can assess multiple treatment strategies for clinical intervention of a patient's condition. The signature may indicate watchfulness or pre-treatment for a side-effect or recommendation for a different treatment.

A professional (e.g., medical professional) can communicate information regarding biomarker analysis to a subject or a subject's family. In some cases, a professional can provide a subject and/or a subject's family with information regarding an IT therapy, including treatment options and potential side effects. In some cases, a professional can provide a copy of a subject's medical records to communicate information regarding biomarker analysis and/or disease states to a specialist.

A professional (e.g., research professional) can apply information regarding a subject's biomarkers to advance research into IT therapy. For example, a researcher can compile data on the presence of a particular signature with information regarding the efficacy of an IT therapy, or side effects associated with an IT therapy. In some cases, a research professional can obtain a subject's biomarker information to evaluate the subject's enrollment, or continued participation in a research study or clinical trial. In some cases, a research professional can communicate a subject's biomarker information to a medical professional, or can refer a subject to a medical professional for clinical assessment and/or treatment.

Any appropriate method can be used to communicate information to another person (e.g., a professional), and information can be communicated directly or indirectly. For example, a laboratory technician can input biomarker information or cancer diagnosis information into a computer-based record. In some cases, information can be communicated by making a physical alteration to medical or research records. For example, a medical professional can make a permanent notation or flag a medical record for communicating information to other medical professionals reviewing the record. Any type of communication can be used (e.g., mail, e-mail, telephone, and face-to-face interactions). Information also can be communicated to a professional by making that information electronically available to the professional. For example, information can be placed on a computer database such that a medical professional can access the information. In addition, information can be communicated to a hospital, clinic, or research facility serving as an agent for the professional.

As used herein, the term “vaccine” means a composition that elicits a prophylactic or therapeutic immune response in a subject. In some cases, the immune response is a protective immune response. Typically, a vaccine elicits an antigen-specific immune response to an antigen of a pathogen, for example, a bacterial or viral pathogen, or to a cellular constituent correlated with a pathological condition, such as cancer. A vaccine may include a polynucleotide, a peptide or polypeptide, a virus, a bacterium, a cell or one or more cellular constituents. In some cases, the virus, bacteria, or cell may be inactivated or attenuated to prevent or reduce the likelihood of infection, while maintaining the immunogenicity of the vaccine constituent. The immunogenic material may include live-attenuated or killed microorganisms (such as bacteria or viruses), or antigenic proteins, peptides or DNA derived from them. In some cases, the vaccine is a subunit vaccine, which is an immunizing agent that has been treated to remove traces of nucleic acid (such as viral nucleic acid) so that only protein subunits remain. The subunits have less risk of causing adverse reactions. The vaccine can also be a live vaccine, which is a vaccine prepared from living attenuated organisms or from viruses that have been attenuated but can still replicate in the cells of the host organism. The immunogenic material for a cancer vaccine may include, for example, a protein or peptide expressed by a tumor or cancer cell. Vaccines may elicit both prophylactic (preventative) and therapeutic responses.

As used herein, the term “vector” means a virus, bacterium, or other microbe, or a nucleic acid, used to deliver an antigen or a gene for an antigen, as part of a vaccine. A nucleic acid vector is a nucleic acid molecule as introduced into a host cell, thereby producing a transformed host cell. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant DNA vectors having at least some nucleic acid sequences derived from one or more viruses.

As used herein, the term “nucleic acid” refers to a polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof, Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (e.g., A, T, G, C), this also includes an RNA sequence (e.g., A, U, G, C) in which “U” replaces “T.” “Nucleotide” includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Conventional notation is used herein to describe nucleotide sequences: the left-hand end of a single-stranded nucleotide sequence is the 5 ‘-end; the left-hand direction of a double-stranded nucleotide sequence is referred to as the 5’-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.” “cDNA” refers to a DNA that is complementary or identical to an mRNA, in cither single stranded or double stranded form.

As used herein, “encode(s)” or “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (for example, rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Recombinant nucleic acid” refers to a nucleic acid having nucleotide sequences that are not naturally joined together. This includes nucleic acid vectors including an amplified or assembled nucleic acid, which can be used to transform a suitable host cell. A host cell that includes the recombinant nucleic acid is referred to as a “recombinant host cell.” The gene is then expressed in the recombinant host cell to produce, such as a “recombinant polypeptide.” A recombinant nucleic acid may serve a non-coding function (such as a promoter, origin of replication, ribosome-binding site, etc.) as well.

As used herein, the term “adjuvant” means a vehicle used to enhance antigenicity; such as a suspension of minerals (alum, aluminum hydroxide, aluminum phosphate) on which antigen is adsorbed; or water-in-oil emulsion in which antigen solution is emulsified in oil (MF-59, Freund's incomplete adjuvant), sometimes with the inclusion of killed mycobacteria (Freund's complete adjuvant) to further enhance antigenicity (inhibits degradation of antigen and/or causes influx of macrophages). Adjuvants also include immunostimulatory molecules, such as cytokines, costimulatory molecules, and for example, immunostimulatory DNA or RNA molecules.

The composition also can be formulated to contain an adjuvant in order to enhance the immunological response. Suitable adjuvants include, but are not limited to, lysolecithin, pluronic polyols, polyanions, other peptides, oil emulsions, and potentially useful human adjuvants such as Bacillus Calmette Guerin (BCG) and Corynebacterium parvum. Adjuvants for inclusion in the inventive composition desirably are safe, well tolerated, such as QS-21, Detox-PC, MPL-SE, MoGM-CSF, TiterMax-G, CRL-1005, GERBU, TERamide, PSC97B, Adjumer, PG-026, GSK-1, GcMAF, B-alethine, MPC-026, Adjuvax, CpG ODN, Betafectin, Alum, and MF59 (as described in, e.g., Kim et al., Vaccine, 18:597 (2000)). Other adjuvants that can be administered to a mammal include lectins, growth factors, cytokines, and lymphokines (e.g., alpha-interferon, gamma-interferon, platelet derived growth factor (PDGF), gCSF, gMCSF, TNF, epidermal growth factor (EGF), IL-1, IL-2, IL-4, IL-6, IL-8, IL-10, and IL-12).ABM2, AS01B, AS02, AS02A, Adjumer, Adjuvax, Algammulin, Alum, Aluminum phosphate, Aluminum potassium sulfate, Bordetella pertussis, Calcitriol, Chitosan, Cholera toxin, CpG, Dibutyl phthalate, Dimethyldioctadecylammonium bromide (DDA), Freund's adjuvant, Freund's complete, Freund's incomplete (IF A), GM-CSF, GMDP, Gamma Inulin, Glycerol, HBSS (Hank's Balanced Salt Solution), Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, and Zymosan.

Cosolvents may be added to a composition or formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters.

Supplementary compounds (e.g., preservatives, antioxidants, antimicrobial agents including biocides and biostats such as antibacterial, antiviral, and antifungal agents) can also be incorporated into the compositions. Pharmaceutical compositions may therefore include preservatives, antioxidants, and antimicrobial agents.

Preservatives can be used to inhibit microbial growth or increase stability of ingredients thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

In certain embodiments, the methods and compositions disclosed herein may include a pharmaceutically acceptable carrier. The pharmaceutically acceptable carriers of use are conventional. Remington's Pharmaceutical Sciences, by E. W. Martin, Mack Publishing Co., Easton, PA, 19th Edition, 1995, describes compositions and formulations suitable for pharmaceutical delivery of the compositions disclosed herein. In general, the nature of the carrier will depend on the particular mode of administration being employed. For instance, parenteral formulations usually include injectable fluids that include pharmaceutically and physiologically acceptable fluids such as water, physiological saline, balanced salt solutions, aqueous dextrose, glycerol, or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Any route of administration can be used to deliver the vaccine composition to the subject. Indeed, although more than one route can be used to administer the composition, a particular route can provide a more immediate and more effective reaction than another route. Exemplary routes of administration for contact or in vivo delivery which a composition can optionally be formulated include inhalation, respiration, intranasal, intubation, intrapulmonary instillation, oral, buccal, intrapulmonary, intradermal, topical, dermal, parenteral, sublingual, subcutaneous, intravascular, intrathecal, intraarticular, intracavity, transdermal, iontophoretic, intraocular, ophthalmic, optical, intravenous (i.v.), intramuscular, intraglandular, intraorgan, or intralymphatic.

Formulations suitable for parenteral administration include aqueous and non-aqueous solutions, suspensions or emulsions of the active compound, which preparations are typically sterile and can be isotonic with the blood of the intended recipient. Non-limiting illustrative examples include water, saline, dextrose, fructose, ethanol, animal, vegetable, or synthetic oils.

In some examples, the composition is administered via intramuscular injection, for example, using a syringe or needleless delivery device. In this respect, this disclosure also provides a syringe or a needleless delivery device including the composition. The composition also can be applied or instilled into body cavities, absorbed through the skin (e.g., via a transdermal patch), inhaled, ingested, topically applied to tissue, or administered parenterally via, for instance, intravenous, peritoneal, or intraarterial administration.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device made of a polymeric composition, are particularly useful for administration of the composition. The composition also can be administered in the form of a sustained-release formulation (see, e.g., U.S. Pat. No. 5,378,475) including, for example, gel foam, hyaluronic acid, gelatin, chondroitin sulfate, a polyphosphoester, such as bis-2-hydroxyethyl-terephthalate (BHET), and/or a polylactic-glycolic acid. It can also be administered using a gene gun via microparticles.

The dose of the composition administered will depend on a number of factors, including the size of a target tissue, the extent of any side-effects, the particular route of administration, and the like. The dose ideally includes an “effective amount” of the composition, e.g., a dose of composition, which provokes a desired immune response in the subject. As used herein, the term “effective amount” includes an amount of agent, such as an agent that is sufficient to generate a desired response, such an immune response. In some examples, an “effective amount” is one that treats (including prophylaxis) one or more symptoms and/or underlying causes of any of a disorder or disease, for example to treat and/or prevent cancer in a subject. In one example, an effective amount is a therapeutically effective amount. In one example, an effective amount is an amount that prevents one or more signs or symptoms of a particular disease or condition from developing, such as one or more signs or symptoms associated with cancer. The desired immune response can entail production of antibodies, protection upon subsequent challenge, immune tolerance, immune cell activation, and the like. One dose or multiple doses of the composition can be administered to a mammal to elicit an immune response with desired characteristics, including the production of specific antibodies, or the production of functional T cells.

Effective dosages and schedules for administering the compositions may be determined empirically, and making such determinations is within the skill in the art. The dosage ranges for the administration of the compositions are those large enough to produce the desired effect in which the symptoms/disorder are/is affected. The dosage should not be so large as to cause adverse side effects, such as unwanted cross-reactions, anaphylactic reactions, and the like. Generally, the dosage will vary with the age, condition, sex, and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 mg/kg to up to 100 mg/kg of body weight or more per day, depending on the factors mentioned above. Following administration of a disclosed composition, such as a vaccine or an antibody, for treating, inhibiting, or preventing a cancer, the efficacy of the therapy or prophylaxis can be assessed in various ways well known to the skilled practitioner. For instance, one of ordinary skill in the art will understand that a composition, such as a vaccine or an antibody, disclosed herein is efficacious in treating, inhibiting, or preventing a cancer in a subject by observing that the composition reduces tumor growth or prevents a further increase in tumor size.

As used herein, “inhibit,” “inhibiting,” and “inhibition” refer to decreasing an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “antibodies” is used herein in a broad sense and includes both polyclonal and monoclonal antibodies. In addition to intact immunoglobulin molecules, also included in the term “antibodies” are fragments or polymers of immunoglobulin molecules, and human or humanized versions of immunoglobulin molecules or fragments thereof. These may be chosen for their ability to interact with tumor-associated ncRNA peptides or other novopeptides or targets of interest, and in embodiments may be used such that tumor growth is inhibited. The antibodies can be tested for their desired activity using the in vitro assays described herein, or by analogous methods, after which their in vivo therapeutic and/or prophylactic activities may be tested according to known clinical testing methods.

As used herein, the term “antibody” encompasses, but is not limited to, whole immunoglobulin (for example, an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (l), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each include four FR regions, largely adopting a beta sheet configuration, connected by three CDRs, which form loops connecting, and in some cases forming part of, the beta sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md.). The constant domains are not involved directly in binding an antibody to an antigen, but exhibit various effector functions, such as participation of the antibody in antibody-dependent cellular toxicity.

As used herein, the term “antibody” or fragments thereof encompasses chimeric antibodies and hybrid antibodies, with dual or multiple antigen or epitope specificities, and fragments, such as F(ab′) 2, Fab′, Fab, sFv, scFv, and the like, including hybrid fragments. Thus, fragments of the antibodies that retain the ability to bind their specific antigens are provided. For example, fragments of antibodies which maintain FS 1-78, FS 6-21, FS SMC1A binding activity are included within the meaning of the term “antibody or fragment thereof” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M J. Curr. Opin. Biotechnol. 3:348-354, 1992).

The term “monoclonal antibody” as used herein refers to an antibody obtained from a substantially homogeneous population of antibodies, for example, the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sd. USA, 81:6851-6855 (1984)).

Administration of the antibodies can be carried out as disclosed herein. Nucleic acid approaches for antibody delivery also exist. Broadly neutralizing antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any operable means, such as, for example, those disclosed herein.

ncRNA Peptide Array

Disclosed herein are arrays of ncRNA peptides. Accordingly, some embodiments provided herein relate to arrays. In some embodiments, the arrays include peptides resulting from tumor specific translation of ncRNA (ncRNAPs). In some embodiments, the arrays include a plurality of ncRNA peptides. In some embodiments, the ncRNAPs on the array are spaced between 3 and 9 μM, such as 3, 4, 5, 6, 7, 8, or 9 μM, or an amount within a range defined by any two of the aforementioned values. In some embodiments, the peptides of an ncRNA peptide array arc 5, 6, 7, 8, 9, 10, 11, 12, 13, 14, 15, 16, 17, 18, 19, or 20 amino acids in length, or a range constructed from any of the aforementioned values. In some embodiments, the peptides are about 10 to about 15 amino acids in length. In some embodiments, an array includes at least one peptide having at least 80%, at least 85%, at least 90%, at least 95%, at least 96%, at least 97%, at least 98%, at least 99%, or 100% sequence identity with any of SEQ ID NOs: 1-17.

The disclosed arrays have a variety of potential uses. In some embodiments, the array is used to identify one or more ncRNAPs of interest, such as ncRNAPs that are immunogenic, cancer-specific, and/or shared across cancers. In such an embodiment, the array may include a large number of ncRNAPs, for example up to 5 million ncRNAPs. This broad coverage of ncRNAPs would allow for the potential of many ncRNAPs to be explored and discovered.

In some embodiments, the array is used to diagnose cancer. In such an embodiment, the array may include ncRNAPs which have been observed across many different cancers, in order to provide a broad panel coverage. In some embodiments, the array may be used to measure immune response to 2 or more peptides of the array, thereby creating a signature which may be used to detect cancer or predict response to immunotherapy. In some embodiments, the arrays are used to predict response to immunotherapy. In some embodiments, the arrays are used to predict adverse responses to immunotherapy. In such an embodiment, the array may include ncRNAPs which have been statistically correlated to a positive (desirable) or negative (undesirable) response to immunotherapy.

In some embodiments, the ncRNAPs are used to design therapeutic or prophylactic vaccines. In some embodiments the arrays are used to determine targets for therapeutics to treat cancer. Certain embodiments of the arrays relate to arrays used in designing a personal cancer vaccine. Certain embodiments of the arrays relate to arrays used in designing a universal cancer vaccine.

In some embodiments, the arrays include at least about 10, about 100, about 200, about 300, about 500, about 1,000, about 2,000, about 3,000, about 5,000, about 7,500, about 10,000, about 12,500, about 15,000, about 17,500, about 20,000, about 22,500, about 25,000, about 27,500, about 30,000, about 32,500, about 35,000, about 37,500, about 40,000, about 50,000, about 100,000, about 200,000, about 300,000, about 400,000, about 500,000, about 600,000, about 700,000, about 800,000, about 900,000, about 1,000,000, about 1,500,000, about 2,000,000, about 2,500,000, about 3,000,000, about 3,500,000, about 4,000,000, about 4,500,000, or about 5,000,000 ncRNAPs, or an amount within a range defined by any two of the aforementioned values. In some embodiments, the arrays include a range of peptides ranging from about 10 to about 2.5M peptides. In some embodiments, a plurality of ncRNA peptides are in-situ synthesized on the array. In some embodiments, the plurality of ncRNA peptides are fixed on a substrate. In some embodiments, the substrate includes glass, silica, composite, resin, or combination thereof.

In some embodiments, the array is configured to detect binding by at least one of fluorescence, luminescence, calorimetry, chromatography, radioactivity, Bio-Layer Interferometry, electro-interference sensing, or surface plasmon resonance. For example, after a biological sample is contacted with the array, binding of the biological sample to the array may be detected and/or measured by at least one of fluorescence, luminescence, calorimetry, chromatography, radioactivity, Bio-Layer Interferometry, electro-interference sensing, or surface plasmon resonance. In some embodiments, the array is configured to facilitate detection of binding by at least one of fluorescence, luminescence, calorimetry, chromatography, radioactivity, Bio-Layer Interferometry, electro-interference sensing, or surface plasmon resonance.

The arrays described herein may be combined with other embodiments. For example, the arrays described may be used in methods of identifying ncRNAPs that are immunogenic, cancer-specific, and/or shared across cancers; methods of detecting cancer; methods of predicting immunotherapy response; methods of designing a cancer vaccine; methods of treating a subject with a vaccine; and methods of producing a therapeutic molecule designed to bind ncRNAPs.

Methods of Identifying ncRNAPs that are Immunogenic, Cancer-specific, and/or Shared Across Cancers

Provided herein are methods of identifying one or more ncRNAPs or one or more nucleic acids encoding ncRNAPs that are immunogenic, cancer-specific, and/or shared across cancers. In some embodiments, the methods include contacting a ncRNA peptide array including a plurality of ncRNAPs with a first biological sample obtained from a first individual known to have a cancer, measuring binding of the first biological sample to the ncRNA peptide array, contacting the same type of ncRNA peptide array with a second biological sample obtained from a second individual, measuring binding of the second biological sample to the ncRNA peptide array, comparing the binding of the two biological samples to the ncRNA peptide array, and identifying one or more ncRNAPs that are immunogenic, cancer-specific, and/or shared across cancers.

As used herein, the term “same type of ncRNA peptide array” refers to two or more ncRNA peptide arrays that include the same ncRNA peptides. The same type of ncRNA peptide array may be the same physical unit, or it may be a different physical unit with the same ncRNA peptides included on the array.

In some embodiments, measuring binding includes detecting antibody reactivity to the plurality of ncRNAPs. In some embodiments, the second individual is a control individual without cancer. Comparison to this control screens out ncRNAPs in which antibody reactivity is normal and unrelated to cancer. In some embodiments, the second individual is also known to have a cancer. In some embodiments, the second individual is known to have the cancer of the first individual. Use of a second individual with the same cancer as the first individual may provide confirmation that a “shared” ncRNAPs (for example, an ncRNAP that is bound by samples of both the first and second individual) is common to a that particular type of cancer. In some embodiments, the second individual and the first individual have different types of cancer. In such an embodiment, comparison of binding between the first individual and the second individual may identify ncRNAPs that are cancer-specific because they are not shared by individuals with different cancers and/or may identify ncRNAPs that are shared across multiple cancers when binding is observed with both the first and second individuals' biological samples. These methods may be combined in that the binding of a first individual may be compared to a second individual who is a control with no cancer, to a third individual who is known to have cancer of the same type as the first individual, and/or a fourth individual who is known to have a different type of cancer. In some embodiments, comparison between groups is quantitative and/or qualitative. In some embodiments, each group (no cancer, same cancer, different cancer) are represented by multiple members.

In some embodiments, the methods further include determining a nucleic acid sequence of said ncRNAPs that are immunogenic to identify one or more nucleic acids encoding ncRNAPs that are immunogenic, cancer-specific, and/or shared across cancers. In some embodiments, determining a nucleic acid sequence of said ncRNAPs that are immunogenic includes identifying one or more ncRNAPs that are immunogenic, cancer-specific, and/or shared across cancers, identifying the amino acid sequence of a ncRNAP, and then working backwards to determine a nucleic acid sequence that encodes said amino acid sequence, according to known mRNA codons which encode amino acids.

In some embodiments, the ncRNA peptide array includes at least about 10, about 100, about 200, about 300, about 500, about 1,000, about 2,000, about 3,000, about 5,000, about 7,500, about 10,000, about 12,500, about 15,000, about 17,500, about 20,000, about 22,500, about 25,000, about 27,500, about 30,000, about 32,500, about 35,000, about 37,500, about 40,000, about 50,000, about 100,000, about 200,000, about 300,000, about 400,000, about 500,000, about 600,000, about 700,000, about 800,000, about 900,000, about 1,000,000, about 1,500,000, about 2,000,000, about 2,500,000, about 3,000,000, about 3,500,000, about 4,000,000, about 4,500,000, or about 5,000,000 ncRNAPs, or an amount within a range defined by any two of the aforementioned values. In some embodiments, the arrays include a range of peptides ranging from about 10 to about 2.5M peptides. In some embodiments, the plurality of ncRNAPs are in-situ synthesized on the array. In some embodiments, the plurality of ncRNAPs are fixed on substrate. In some embodiments, the substrate includes glass, silica, composite, resin, or combination thereof. In some embodiments, binding is detected by at least one of fluorescence, luminescence, calorimetry, chromatography, radioactivity, Bio-Layer Interferometry, electro-interference sensing, or surface plasmon resonance.

The methods of identifying one or more ncRNAPs or one or more nucleic acids encoding ncRNAPs that are immunogenic, cancer-specific, and/or shared across cancers may be combined with other methods and compositions described herein. For example, ncRNAPs and related nucleic acids identified by the methods described herein may be used to create diagnostic arrays, immunotherapy response prediction arrays, vaccine compositions, and personalized vaccine design arrays. ncRNAPs and related nucleic acids identified by the methods described herein may be used in other methods described herein, such as methods of detecting cancer, methods of predicting response to immune therapy, methods of designing a cancer vaccine, methods of treatment/prevention via a cancer vaccine, methods of treatment via a therapeutic molecule, and methods of producing therapeutic antibodies designed to bind an ncRNAP.

In a further embodiment, disclosed herein are methods of measuring an immune response to a plurality of ncRNA peptides. In some embodiments, the method includes a) contacting a biological sample with an ncRNA peptide array comprising a plurality of ncRNA peptides, and b) measuring binding of the biological sample to the ncRNA peptide array. The ncRNA peptide array may be an array as further described herein. Measuring binding may be performed as further described herein. In some embodiments, the ncRNA peptide array comprises at least one peptide having at least 95% sequence identity with any of SEQ ID NOs: 1-17.

### Methods of Detecting Cancer

Provided herein are methods of detecting cancer in a subject. In some embodiments, the methods include obtaining a biological sample from a subject, contacting the biological sample from the subject with an array including a plurality of ncRNAPs, measuring binding of the biological sample to the plurality of ncRNAPs, and analyzing the binding to predict whether the subject has cancer. In some embodiments, the ncRNAPs include one or more peptides discovered by methods described in Zhao et al. (Translation of noncoding RNAs and cancer, Cancer Letters 497 (2021) 89-99, which is incorporated by reference in its entirety). In some embodiments, the ncRNAPs result from non-conventional translation of a ncRNA.

In some embodiments, measuring binding includes detecting antibody reactivity to at least one peptide of the array. In some embodiments, binding is detected by at least one of fluorescence, luminescence, calorimetry, chromatography, radioactivity, electro-interference, Bio-Layer Interferometry, electro-interference sensing, or surface plasmon resonance.

In some embodiments, analyzing the binding to predict whether the subject has cancer includes comparing the binding to previously-measured binding patterns of other subjects who are known to have had cancer, or other subjects who did not have cancer (for example, positive and negative controls). For example, in some embodiments, binding is compared quantitatively and/or qualitatively to binding of a second subject, who is known to have cancer, to the same type of array. Sufficient similarity of binding is associated with having cancer, and it is predicted that the first subject has cancer. In some embodiments, the array may be used to measure immune response to 2 or more peptides of the array, thereby creating a signature which may be used to detect cancer.

In some embodiments, the plurality of ncRNAPs are fixed on a substrate. In some embodiments, the substrate includes glass, silica, composite, resin, or combination thereof. In some embodiments, the plurality of ncRNAPs are in-situ synthesized on the array. In some embodiments, the array includes at least about 10, about 100, about 200, about 300, about 500, about 1,000, about 2,000, about 3,000, about 5,000, about 7,500, about 10,000, about 12,500, about 15,000, about 17,500, about 20,000, about 22,500, about 25,000, about 27,500, about 30,000, about 32,500, about 35,000, about 37,500, about 40,000, about 50,000, about 100,000, about 200,000, about 300,000, about 400,000, about 500,000, about 600,000, about 700,000, about 800,000, about 900,000, about 1,000,000, about 1,500,000, about 2,000,000, about 2,500,000, about 3,000,000, about 3,500,000, about 4,000,000, about 4,500,000, or about 5,000,000 ncRNAPs, or an amount within a range defined by any two of the aforementioned values. In some embodiments, the arrays include a range of peptides ranging from about 10 to about 2.5M peptides.

In some embodiments, the biological sample includes blood, serum, plasma, cerebrospinal fluid, saliva, urine, or combinations thereof. In some embodiments, the biological sample includes antibodies. In some embodiments, the subject is a mammal. In some embodiments, the subject is a human, a dog, a cat, a mouse, a rat, a rabbit, a horse, a cow, or a pig.

In some embodiments, the subject is suspected of having a cancer. In some embodiments, the cancer is selected from the group consisting of acute lymphoblastic leukemia, acute monocytic leukemia, acute myeloid leukemia, acute promyelocytic leukemia, adenocarcinoma, adult T-cell leukemia, astrocytoma, bladder cancer, bone cancer, brain tumor, breast cancer, Burkitt's lymphoma, carcinoma, cervical cancer, chronic lymphocytic leukemia, chronic myelogenous leukemia, colon cancer, colorectal cancer, endometrial cancer, glioblastoma multiforme, glioma, hepatocellular carcinoma, Hodgkin's lymphoma, inflammatory breast cancer, kidney cancer, leukemia, lung cancer, lymphoma, malignant mesothelioma, medulloblastoma, melanoma, multiple myeloma, neuroblastoma, non-Hodgkin lymphoma, non-small cell lung cancer, ovarian cancer, pancreatic cancer, pituitary tumor, prostate cancer, retinoblastoma, skin cancer, small cell lung cancer, squamous cell carcinoma, stomach cancer, T-cell leukemia, T-cell lymphoma, thyroid cancer, and Wilms' tumor.

Methods disclosed herein may be combined with other methods. For example, a subject's biological sample may be contacted with an ncRNA peptide array, and binding of the sample to one or more peptides of the ncRNA array may be used to simultaneously detect cancer, predict response to immunotherapy, identify immunogenic peptides for use in a preventative or therapeutic vaccine, and/or identify antibodies for use in treatment with a therapeutic molecule designed to bind an ncRNAP. In some embodiments, the ncRNA peptide array may include ncRNA peptides which have previously been identified as being immunogenic, cancer-specific, or shared across cancers according to the methods disclosed herein.

### Methods of Predicting Response to Immunotherapy

Disclosed herein are methods of predicting the response of a subject with cancer to an immunotherapy. In some embodiments, the methods include obtaining a biological sample from a subject with cancer, contacting the biological sample to an array including a plurality of ncRNAPs, and measuring binding of the biological sample to the plurality of ncRNAPs. In some embodiments, measuring binding includes detecting antibody reactivity to at least one peptide of the array.

In some embodiments, the methods further include predicting a response of the subject to an immunotherapy. In some embodiments, the methods further include analyzing the binding to predict whether immunotherapy would be effective in treating the subject's cancer. In some embodiments, the methods further include analyzing the binding to predict whether immunotherapy would elicit an adverse immune response to immunotherapy in the subject. In some embodiments, comparison is quantitative and/or qualitative. In some embodiments, analyzing includes comparing the binding of the biological sample to binding of another subject who responded positively to immunotherapy or experienced an adverse immune response in response to immunotherapy. In some embodiments, the binding is compared to another subject who had no response to immunotherapy (for example, immunotherapy was not effective in treating the subject's cancer). In some embodiments the binding is compared to another subject who did not have an adverse event in response to an immunotherapy. In some embodiments, based on this analysis, the subject is classified as being likely to respond (positively) to treatment with the immunotherapeutic, or as being likely to have an adverse event in response to immunotherapy.

Certain embodiments relate to methods of predicting the response of a subject with cancer to an immunotherapy, and as described herein, can include, for example, obtaining the subject's binding signature to an ncRNAPs array using one or more biological samples obtained from the subject to determine whether the sample contains one or more indicators of favorable or unfavorable responses (e.g., unfavorable side effects) to IT therapy. As used herein, “binding signature,” “binding profile,” or “ncRNAP signature” refers the observed binding of a subject's biological sample to a ncRNAPs array. A binding signature or binding profile may include qualitative information about whether a subject's biological sample was observed to bind to a particular ncRNA peptide on an ncRNA peptide array and may include quantitative information such as the level or strength of observed binding to particular ncRNA peptides on an ncRNA peptide array. The correlation between a binding signature or binding profile and responsiveness to IT therapy can be established by obtaining binding signatures for subjects having a known favorable response to IT treatment and for subjects that were unresponsive or had an unfavorable response to treatment using sera (or other bodily samples) collected before each subject received treatment. In some cases, a control includes non-disease sera contacted with an identical array under the same experimental conditions. The breadth of the binding profile can be quantified in multiple ways including, for example the number of motifs, the percentage of signature represented, and/or total immune reactivity. Once the quantitative correlate has been established, cancer patients can be classified according to a method provided herein by quantifying a subject's signature for responsiveness to IT treatment, prognosis, or likelihood of experiencing serious side-effects of IT treatment. In such cases, the methods are useful for determining a subject's responsiveness for IT treatment of a tumor (including early-stage tumor formation) associated with ncRNA peptide expression.

An ncRNAP signature is established by using a biological sample (e.g., blood, sera, plasma) that may contain antibodies having affinity to peptides on the ncRNAP array. As further described herein, antibody reactivity to at least one peptide of the array may be detected. As described herein, antibodies are employed as biomarkers of disease, thus taking advantage of the immune system's expansive antibody repertoire to identify a statistically significant pattern of peptides, each with specific binding values having predictive, prognostic, and diagnostic potential. In some cases, the biological sample is diluted. The sample is incubated long enough to allow cognate binding to approach equilibrium-usually overnight. The array is washed and then incubated with secondary antibody to quantify the amount of antibody bound to each peptide on the array. For each peptide, a quantitative amount of fluorescence is determined. These quantitative data can be analyzed in many different analytical and statistical approaches. In general, a patient's ncRNAP signature for IT response, prognosis, or side-effects is determined by comparing two or more groups of interest. For example, a comparison may be made between patients who responded well to IT therapy and those that did not. Such comparisons are used to establish the classifier of interest. In some cases, because the ncRNAP arrays are directly measuring the immune response to tumor antigens, the difference in groups may be determined directly by quantifying total binding to the ncRNA peptides. In some embodiments, the array may be used to measure immune response to 2 or more peptides of the array, thereby creating a signature which may be used to predict response to immunotherapy.

In some embodiments, the plurality of ncRNAPs are fixed on a substrate. In some embodiments, the substrate includes glass, silica, composite, resin, or combination thereof. In some embodiments, the plurality of ncRNAPs are in-situ synthesized on the array. In some embodiments, binding is detected by at least one of fluorescence, luminescence, calorimetry, chromatography, radioactivity, electro-interference, Bio-Layer Interferometry, electro-interference sensing, or surface plasmon resonance. In some embodiments, the array includes at least about 10, about 100, about 200, about 300, about 500, about 1,000, about 2,000, about 3,000, about 5,000, about 7,500, about 10,000, about 12,500, about 15,000, about 17,500, about 20,000, about 22,500, about 25,000, about 27,500, about 30,000, about 32,500, about 35,000, about 37,500, about 40,000, about 50,000, about 100,000, about 200,000, about 300,000, about 400,000, about 500,000, about 600,000, about 700,000, about 800,000, about 900,000, about 1,000,000, about 1,500,000, about 2,000,000, about 2,500,000, about 3,000,000, about 3,500,000, about 4,000,000, about 4,500,000, or about 5,000,000 ncRNAPs, or an amount within a range defined by any two of the aforementioned values. In some embodiments, the arrays include a range of peptides ranging from about 10 to about 2.5M peptides. In some embodiments, the biological sample includes blood, serum, plasma, cerebrospinal fluid, saliva, urine, or combinations thereof. In some embodiments, the biological sample includes antibodies. In some embodiments, the subject is a mammal. In some embodiments, the subject is a human, a dog, a cat, a mouse, a rat, a rabbit, a horse, a cow, or a pig.

In some embodiments, the cancer is selected from the group consisting of acute lymphoblastic leukemia, acute monocytic leukemia, acute myeloid leukemia, acute promyelocytic leukemia, adenocarcinoma, adult T-cell leukemia, astrocytoma, bladder cancer, bone cancer, brain tumor, breast cancer, Burkitt's lymphoma, carcinoma, cervical cancer, chronic lymphocytic leukemia, chronic myelogenous leukemia, colon cancer, colorectal cancer, endometrial cancer, glioblastoma multiforme, glioma, hepatocellular carcinoma, Hodgkin's lymphoma, inflammatory breast cancer, kidney cancer, leukemia, lung cancer, lymphoma, malignant mesothelioma, medulloblastoma, melanoma, multiple myeloma, neuroblastoma, non-Hodgkin lymphoma, non-small cell lung cancer, ovarian cancer, pancreatic cancer, pituitary tumor, prostate cancer, retinoblastoma, skin cancer, small cell lung cancer, squamous cell carcinoma, stomach cancer, T-cell leukemia, T-cell lymphoma, thyroid cancer, and Wilms' tumor.

Methods of predicting response to an immunotherapy may be combined with other methods and embodiments disclosed herein. In some embodiments, the array may include a plurality of ncRNA peptides which have previously been identified as being immunogenic, cancer-specific, or shared across cancers according to the methods disclosed herein.

### Vaccine Composition

Disclosed herein are vaccine compositions. In certain embodiments, the vaccine compositions include one or more ncRNA peptides. In some embodiments, biological samples, such as blood, from cancer patients are applied to the ncRNAP arrays described herein to determine reactivity of peptides for each patient. In some embodiments, ncRNAPs unique to the patient are used in a personal vaccine. In some embodiments, ncRNAPs shared between different patients are used for off-the-shelf therapeutic or preventative vaccines. In some embodiments, the vaccine compositions include ncRNAPs resulting from translation of ncRNA. In some embodiments, the vaccine composition includes two or more pooled ncRNAPs.

In some embodiments, the vaccine compositions further include an adjuvant. In some embodiments, the adjuvant is ABM2, AS01B, AS02, AS02A, Adjumer, Adjuvax, Algammulin, alum, aluminum phosphate, aluminum potassium sulfate, Bordetella pertussis, calcitriol, chitosan, cholera toxin, CpG, dibutyl phthalate, dimethyldioctadecylammonium bromide (DDA), Freund's adjuvant, Freund's complete, Freund's incomplete (IFA), GM-CSF, GMDP, gamma inulin, glycerol, HBSS (Hank's Balanced Salt Solution), polyinosinic-polycytidylic acid stabilized with polylysine and carboxymethylcellulose (poly-ICLC, also known as Hiltonol), IL-12, IL-2, imiquimod, interferon-gamma, ISCOM, lipid core peptide (LCP), lipofectin, lipopolysaccharide (LPS), liposomes, MF59, MLP+TDM, monophosphoryl lipid A, Montanide IMS-1313, Montanide ISA 206, Montanide ISA 720, Montanide ISA-51, Montanide ISA-50, nor-MDP, oil-in-water emulsion, P1005 (non-ionic copolymer), Pam3Cys (lipoprotein), pertussis toxin, poloxamer, QS21, RaLPS, Ribi, saponin, Seppic ISA 720, soybean oil, squalene, Syntex adjuvant formulation (SAF), synthetic polynucleotides (poly IC/poly AU), TiterMax tomatine, Vaxfectin, XtendIII, or Zymosan.

### Methods of Designing a Cancer Vaccine

Disclosed herein are methods of designing a cancer vaccine. In some embodiments, the methods include identifying one or more ncRNAPs or one or more nucleic acids encoding ncRNAPs that are immunogenic and preparing a vaccine including one or more ncRNAPs or one or more nucleic acids encoding ncRNAPs that are immunogenic.

In some embodiments, identifying one or more ncRNAPs or one or more nucleic acids encoding ncRNAPs that are immunogenic includes contacting a ncRNA peptide array including one or more ncRNAPs with a first biological sample obtained from a first individual known to have a cancer, measuring binding of the first biological sample to the ncRNA peptide array, contacting the same type of ncRNA peptide array with a second biological sample obtained from a second individual, measuring binding of the second biological sample to the ncRNA peptide array, comparing the binding of the two biological samples to the ncRNA peptide array, and identifying one or more ncRNAPs that are immunogenic.

In some embodiments, the methods further include determining a nucleic acid sequence of said ncRNAPs that are immunogenic to identify one or more nucleic acids encoding ncRNAPs that are immunogenic. In some embodiments, determining a nucleic acid sequence of said ncRNAPs that are immunogenic includes identifying an ncRNAPs that is immunogenic, identifying the amino acid sequence of the ncRNAP, and then working backwards to determine a nucleic acid sequence that encodes said amino acid sequence, according to known mRNA codons which encode amino acids.

In some embodiments, measuring binding includes detecting antibody reactivity to the plurality of ncRNAPs. In some embodiments, the ncRNA peptide array binding is detected by at least one of fluorescence, luminescence, calorimetry, chromatography, radioactivity, electro-interference, Bio-Layer Interferometry, electro-interference sensing, or surface plasmon resonance.

In some embodiments, the second individual is a control individual without cancer. Comparison to this control screens out ncRNAPs in which antibody reactivity is normal and unrelated to cancer. In some embodiments, the second individual is also known to have a cancer. In some embodiments, the second individual is known to have the cancer of the first individual. Use of a second individual with the same cancer as the first individual may provide confirmation that a “shared” ncRNAPs (for example, an ncRNAP that is bound by samples of both the first and second individual) is common to a that particular type of cancer. In some embodiments, the second individual and the first individual have different types of cancer. In such an embodiment, comparison of binding between the first individual and the second individual may identify ncRNAPs that are cancer-specific because they are not shared by individuals with different cancers and/or may identify ncRNAPs that are shared across multiple cancers when binding is observed with both the first and second individuals' biological samples. These methods may be combined in that the binding of a first individual may be compared to a second individual who is a control with no cancer, to a third individual who is known to have cancer of the same type as the first individual, and/or a fourth individual who is known to have a different type of cancer. In some embodiments, each group (no cancer, same cancer, different cancer) would be represented by multiple members.

In some embodiments, the plurality of ncRNAPs is fixed on a substrate. In some embodiments, the substrate includes glass, silica, composite, resin, or combination thereof. In some embodiments, the plurality of ncRNAPs is in-situ synthesized on the array. In some embodiments, the ncRNA peptide array includes at least about 10, about 100, about 200, about 300, about 500, about 1,000, about 2,000, about 3,000, about 5,000, about 7,500, about 10,000, about 12,500, about 15,000, about 17,500, about 20,000, about 22,500, about 25,000, about 27,500, about 30,000, about 32,500, about 35,000, about 37,500, about 40,000, about 50,000, about 100,000, about 200,000, about 300,000, about 400,000, about 500,000, about 600,000, about 700,000, about 800,000, about 900,000, about 1,000,000, about 1,500,000, about 2,000,000, about 2,500,000, about 3,000,000, about 3,500,000, about 4,000,000, about 4,500,000, or about 5,000,000 ncRNAPs, or an amount within a range defined by any two of the aforementioned values. In some embodiments, the arrays include a range of peptides ranging from about 10 to about 2.5M peptides.

In some embodiments, the vaccine compositions further include an adjuvant. In some embodiments, the adjuvant is ABM2, AS01B, AS02, AS02A, Adjumer, Adjuvax, Algammulin, alum, aluminum phosphate, aluminum potassium sulfate, Bordetella pertussis, calcitriol, chitosan, cholera toxin, CpG, dibutyl phthalate, dimethyldioctadecylammonium bromide (DDA), Freund's adjuvant, Freund's complete, Freund's incomplete (IFA), GM-CSF, GMDP, gamma inulin, glycerol, HBSS (Hank's Balanced Salt Solution), polyinosinic-polycytidylic acid stabilized with polylysine and carboxymethylcellulose (poly-ICLC, also known as Hiltonol), IL-12, IL-2, imiquimod, interferon-gamma, ISCOM, lipid core peptide (LCP), lipofectin, lipopolysaccharide (LPS), liposomes, MF59, MLP+TDM, monophosphoryl lipid A, Montanide IMS-1313, Montanide ISA 206, Montanide ISA 720, Montanide ISA-51, Montanide ISA-50, nor-MDP, oil-in-water emulsion, P1005 (non-ionic copolymer), Pam3Cys (lipoprotein), pertussis toxin, poloxamer, QS21, RaLPS, Ribi, saponin, Seppic ISA 720, soybean oil, squalene, Syntex adjuvant formulation (SAF), synthetic polynucleotides (poly IC/poly AU), TiterMax tomatine, Vaxfectin, XtendIII, or Zymosan.

These methods may be combined with other embodiments disclosed herein. For example, in some embodiments, the vaccine is designed based at least in part on ncRNA peptides that were identified as immunoreactive when the subject's biological sample was contacted to an ncRNA peptide diagnostic array. In some embodiments, the subject's biological sample is immunoreactive with an ncRNA peptide of a diagnostic array as described herein, and a cancer vaccine is designed including that ncRNA peptide. In another example, methods of vaccine design may be combined with methods of treatment/prevention when the vaccine is designed, produced, and then administered to a subject.

In a further example, methods of detection, vaccine design, and treatment/prevention are combined when a biological sample from a subject is contacted with an array of ncRNA peptides, binding is observed between the sample and an ncRNA peptide, and cancer is thereby detected. Then, a vaccine is designed using the ncRNA peptide identified with the array, and the vaccine is administered to the subject to treat/prevent cancer.

The methods of designing a cancer vaccine may be combined with the methods of identifying ncRNAPs that are immunogenic, cancer-specific, and/or shared across cancers disclosed herein. For example, methods disclosed herein are used to identify an ncRNAP that is immunogenic and shared across cancers. Then, a cancer vaccine is designed including an ncRNAP that is immunogenic and shared across cancers. In another embodiment, methods disclosed herein are used to identify an ncRNAP that is immunogenic and cancer-specific, and a cancer vaccine is designed including an ncRNAP that is immunogenic and cancer-specific.

### Methods of Treatment/Prevention Via Universal Vaccine

Disclosed herein are methods for treating or preventing cancer, including administering a vaccine including a) one or more ncRNA peptides or b) a nucleic acid sequence encoding one or more ncRNA peptides. In some embodiments, the one or more ncRNA peptides have been shown to be immunogenic, cancer-specific, tumor-specific, and/or shared across cancers.

In some embodiments, the one or more ncRNA peptides are identified as being immunogenic, cancer-specific, and/or shared across cancers through the disclosed methods. In some embodiments, the one or more ncRNA peptides of the vaccine composition were identified as being immunogenic, cancer-specific, and/or shared across cancers by contacting a ncRNA peptide array including a plurality of ncRNAPs with a first biological sample obtained from a first individual known to have a cancer, measuring binding of the first biological sample to the ncRNA peptide array, contacting the same type of ncRNA peptide array with a second biological sample obtained from a second individual, measuring binding of the second biological sample to the ncRNA peptide array, comparing the binding of the two biological samples to the ncRNA peptide array, and identifying one or more ncRNAPs that are immunogenic, cancer-specific, and/or shared across cancers.

In some embodiments, the nucleic acid sequence is identified as encoding one or more immunogenic ncRNA peptides. In some embodiments, determining a nucleic acid sequence of said ncRNAPs that are immunogenic includes identifying an ncRNAPs that is immunogenic, identifying the amino acid sequence of the ncRNAP, and then working backwards to determine a nucleic acid sequence that encodes said amino acid sequence, according to known mRNA codons which encode amino acids.

In some embodiments, the vaccine further includes an adjuvant. In some embodiments, the adjuvant is ABM2, AS01B, AS02, AS02A, Adjumer, Adjuvax, Algammulin, alum, aluminum phosphate, aluminum potassium sulfate, Bordetella pertussis, calcitriol, chitosan, cholera toxin, CpG, dibutyl phthalate, dimethyldioctadecylammonium bromide (DDA), Freund's adjuvant, Freund's complete, Freund's incomplete (IFA), GM-CSF, GMDP, gamma inulin, glycerol, HBSS (Hank's Balanced Salt Solution), polyinosinic-polycytidylic acid stabilized with polylysine and carboxymethylcellulose (poly-ICLC, also known as Hiltonol), IL-12, IL-2, imiquimod, interferon-gamma, ISCOM, lipid core peptide (LCP), lipofectin, lipopolysaccharide (LPS), liposomes, MF59, MLP+TDM, monophosphoryl lipid A, Montanide IMS-1313, Montanide ISA 206, Montanide ISA 720, Montanide ISA-51, Montanide ISA-50, nor-MDP, oil-in-water emulsion, P1005 (non-ionic copolymer), Pam3Cys (lipoprotein), pertussis toxin, poloxamer, QS21, RaLPS, Ribi, saponin, Seppic ISA 720, soybean oil, squalene, Syntex adjuvant formulation (SAF), synthetic polynucleotides (poly IC/poly AU), TiterMax tomatine, Vaxfectin, XtendIII, or Zymosan.

In some embodiments, the vaccine is administered to a mammal. In some embodiments, the vaccine is administered to a human, a dog, a cat, a mouse, a rat, a rabbit, a horse, a cow, or a pig. In some embodiments, the cancer is selected from the group consisting of acute lymphoblastic leukemia, acute monocytic leukemia, acute myeloid leukemia, acute promyelocytic leukemia, adenocarcinoma, adult T-cell leukemia, astrocytoma, bladder cancer, bone cancer, brain tumor, breast cancer, Burkitt's lymphoma, carcinoma, cervical cancer, chronic lymphocytic leukemia, chronic myelogenous leukemia, colon cancer, colorectal cancer, endometrial cancer, glioblastoma multiforme, glioma, hepatocellular carcinoma, Hodgkin's lymphoma, inflammatory breast cancer, kidney cancer, leukemia, lung cancer, lymphoma, malignant mesothelioma, medulloblastoma, melanoma, multiple myeloma, neuroblastoma, non-Hodgkin lymphoma, non-small cell lung cancer, ovarian cancer, pancreatic cancer, pituitary tumor, prostate cancer, retinoblastoma, skin cancer, small cell lung cancer, squamous cell carcinoma, stomach cancer, T-cell leukemia, T-cell lymphoma, thyroid cancer, and Wilms' tumor. In some embodiments, treating the cancer includes reducing tumor size, inhibiting tumor growth, reducing tumor burden, increasing survival, or increasing cancer-free survival. In some embodiments, administering the vaccine elicits an immune response in the subject against the cancer.

In some embodiments, the vaccine composition is administered in combination with a therapeutic or preventative antibody as disclosed herein. In some embodiments, the vaccine is administered after cancer has been detected in the subject using an ncRNA array as described herein. For example, a biological sample from a subject is contacted with an array of ncRNA peptides, binding is observed between the sample and an ncRNA peptide, and cancer is thereby detected. Then, a vaccine including one or more ncRNA peptides or a nucleic acid sequence encoding one or more ncRNA peptides is administered to the subject to treat and/or prevent the cancer.

The methods of treatment via a vaccine may be combined with methods disclosed herein relating to designing a cancer vaccine. For example, a vaccine including ncRNAPs that are immunogenic, cancer-specific, or shared across cancers is designed according to methods disclosed herein. Then, the vaccine is administered according to the methods of treatment disclosed herein.

### Methods of Treatment Via Personalized Vaccine

Disclosed herein are methods for treating a subject in need of treatment for a cancer. In some embodiments, the methods include a) obtaining a biological sample from the subject, b) identifying one or more ncRNA peptides that are immunoreactive with a biological sample from the subject in a first population of ncRNA peptides, c) preparing a vaccine composition including a second population of ncRNA peptides including one or more peptides identified in step b) or a nucleic acid sequence encoding the second population of peptides, and d) administering an effective amount of the vaccine composition to the subject, thereby treating the cancer.

In some embodiments, identifying one or more ncRNA peptides that are immunoreactive includes contacting the biological sample with an array including a first population of ncRNA peptides, measuring binding of the biological sample to the first population of ncRNA peptides of the array, and determining a second population of ncRNA peptides of the array that are immunoreactive with the biological sample. In some embodiments, the second population of ncRNA peptides is a subpopulation of the first population of ncRNA peptides.

As used herein, the term “immunoreactive” includes peptides which, when contacted with the subject's biological sample, are bound by the subject's antibodies. “Immunoreactive” also includes peptides which, when administered to a subject, provoke an immune response in the subject.

In some embodiments, the methods further include determining a nucleic acid sequence of said ncRNA peptides to identify one or more nucleic acids encoding ncRNA peptides that are immunoreactive. In some embodiments, determining a nucleic acid sequence of said ncRNAPs that are immunogenic includes identifying an ncRNAPs that is immunoreactive, identifying the amino acid sequence of the ncRNAP, and then working backwards to determine a nucleic acid sequence that encodes said amino acid sequence, according to known mRNA codons which encode amino acids.

In some embodiments, the subject is a mammal. In some embodiments, the subject is a human, a dog, a cat, a mouse, a rat, a rabbit, a horse, a cow, or a pig. In some embodiments, the biological sample is selected from the group consisting of blood, plasma, serum, thymus, bone marrow, spleen, lymph node, bronchoalveolar lavage, breast, central nervous system, cerebrospinal fluid, eye, tears, gastrointestinal tract, saliva, feces, urine, heart, kidney, liver, lung, muscle, pancreas, peripheral nervous system, saliva, skin, thyroid, trachea, and tumor. In some embodiments, the biological sample includes an antibody.

In some embodiments, the cancer is selected from the group consisting of acute lymphoblastic leukemia, acute monocytic leukemia, acute myeloid leukemia, acute promyelocytic leukemia, adenocarcinoma, adult T-cell leukemia, astrocytoma, bladder cancer, bone cancer, brain tumor, breast cancer, Burkitt's lymphoma, carcinoma, cervical cancer, chronic lymphocytic leukemia, chronic myelogenous leukemia, colon cancer, colorectal cancer, endometrial cancer, glioblastoma multiforme, glioma, hepatocellular carcinoma, Hodgkin's lymphoma, inflammatory breast cancer, kidney cancer, leukemia, lung cancer, lymphoma, malignant mesothelioma, medulloblastoma, melanoma, multiple myeloma, neuroblastoma, non-Hodgkin lymphoma, non-small cell lung cancer, ovarian cancer, pancreatic cancer, pituitary tumor, prostate cancer, retinoblastoma, skin cancer, small cell lung cancer, squamous cell carcinoma, stomach cancer, T-cell leukemia, T-cell lymphoma, thyroid cancer, and Wilms' tumor. In some embodiments, treating the cancer includes reducing tumor size, inhibiting tumor growth, reducing tumor burden, increasing survival, or increasing cancer-free survival. In some embodiments, administering the vaccine composition elicits an immune response in the subject against the cancer.

In some embodiments, the vaccine composition further includes a pharmaceutically acceptable adjuvant or excipient. In some embodiments, the adjuvant is ABM2, AS01B, AS02, AS02A, Adjumer, Adjuvax, Algammulin, alum, aluminum phosphate, aluminum potassium sulfate, Bordetella pertussis, calcitriol, chitosan, cholera toxin, CpG, dibutyl phthalate, dimethyldioctadecylammonium bromide (DDA), Freund's adjuvant, Freund's complete, Freund's incomplete (IFA), GM-CSF, GMDP, gamma inulin, glycerol, HBSS (Hank's Balanced Salt Solution), polyinosinic-polycytidylic acid stabilized with polylysine and carboxymethylcellulose (poly-ICLC, also known as Hiltonol), IL-12, IL-2, imiquimod, interferon-gamma, ISCOM, lipid core peptide (LCP), lipofectin, lipopolysaccharide (LPS), liposomes, MF59, MLP+TDM, monophosphoryl lipid A, Montanide IMS-1313, Montanide ISA 206, Montanide ISA 720, Montanide ISA-51, Montanide ISA-50, nor-MDP, oil-in-water emulsion, P1005 (non-ionic copolymer), Pam3Cys (lipoprotein), pertussis toxin, poloxamer, QS21, RaLPS, Ribi, saponin, Seppic ISA 720, soybean oil, squalene, Syntex adjuvant formulation (SAF), synthetic polynucleotides (poly IC/poly AU), TiterMax tomatine, Vaxfectin, XtendIII, or Zymosan.

Methods described herein may be combined with other embodiments described herein. For example, the arrays of the methods described herein may also be used in methods of detecting cancer and/or predicting immune response to immunotherapy. A biological sample from a subject is contacted with an ncRNA peptide array, and binding of the sample to the array is measured. Binding of the sample to an ncRNAP of the array is used to detect cancer and predict an immune response to immunotherapy. A vaccine is prepared based on an ncRNAP identified as immunoreactive with the array. The vaccine is administered to the subject to treat/prevent cancer growth in the subject. In some embodiments, the methods of treatment/prevention with a vaccine described herein may be combined with methods of treatment/prevention with a therapeutic molecule designed to bind an ncRNAP. In some embodiments, the array may include ncRNA peptides which have previously been identified as being immunogenic, cancer-specific, or shared across cancers according to the methods disclosed herein.

### Methods of Treatment/Prevention Via Therapeutic Molecule

Disclosed herein are methods of treating or preventing cancer. In some embodiments, the methods include administering a therapeutic molecule designed to bind a ncRNAP. In some embodiments, the ncRNAP is presented by a tumor. In some embodiments, the therapeutic molecule is an antibody or synthetic antibody. In some embodiments, the therapeutic molecule is administered in combination with another cancer treatment.

In some embodiments, the cancer is selected from the group consisting of acute lymphoblastic leukemia, acute monocytic leukemia, acute myeloid leukemia, acute promyelocytic leukemia, adenocarcinoma, adult T-cell leukemia, astrocytoma, bladder cancer, bone cancer, brain tumor, breast cancer, Burkitt's lymphoma, carcinoma, cervical cancer, chronic lymphocytic leukemia, chronic myelogenous leukemia, colon cancer, colorectal cancer, endometrial cancer, glioblastoma multiforme, glioma, hepatocellular carcinoma, Hodgkin's lymphoma, inflammatory breast cancer, kidney cancer, leukemia, lung cancer, lymphoma, malignant mesothelioma, medulloblastoma, melanoma, multiple myeloma, neuroblastoma, non-Hodgkin lymphoma, non-small cell lung cancer, ovarian cancer, pancreatic cancer, pituitary tumor, prostate cancer, retinoblastoma, skin cancer, small cell lung cancer, squamous cell carcinoma, stomach cancer, T-cell leukemia, T-cell lymphoma, thyroid cancer, and Wilms' tumor. In some embodiments, the therapeutic molecule is administered to a mammal. In some embodiments, the therapeutic molecule is administered to a human, a dog, a cat, a mouse, a rat, a rabbit, a horse, a cow, or a pig.

In some embodiments, treating the cancer includes reducing tumor size, inhibiting tumor growth, reducing tumor burden, increasing survival, or increasing cancer-free survival. In some embodiments, administering the therapeutic molecule elicits an immune response in the subject against the cancer.

In some embodiments, the methods of treatment/prevention with a therapeutic molecule designed to bind an ncRNAP described herein may be combined with methods of treatment/prevention with a vaccine.

### Method of Producing Therapeutic or Preventative Antibody

Disclosed herein are methods of producing a therapeutic or preventative antibody specific for cancer of interest. In some embodiments, the methods include contacting one or more biological samples obtained from one or more subjects identified as having the cancer of interest to an array including ncRNA peptides produced by one or more tumors; detecting antibodies which bind to the ncRNA peptides; selecting one or more ncRNA peptides that are immunoreactive with the antibodies; and preparing an antibody composition against the selected ncRNA peptides for the cancer of interest.

In some embodiments, the ncRNA peptides of the array are spaced between 3 and 9 μM apart. In some embodiments, the array includes at least about 10, about 100, about 200, about 300, about 500, about 1,000, about 2,000, about 3,000, about 5,000, about 7,500, about 10,000, about 12,500, about 15,000, about 17,500, about 20,000, about 22,500, about 25,000, about 27,500, about 30,000, about 32,500, about 35,000, about 37,500, about 40,000, about 50,000, about 100,000, about 200,000, about 300,000, about 400,000, about 500,000, about 600,000, about 700,000, about 800,000, about 900,000, about 1,000,000, about 1,500,000, about 2,000,000, about 2,500,000, about 3,000,000, about 3,500,000, about 4,000,000, about 4,500,000, or about 5,000,000 ncRNAPs, or an amount within a range defined by any two of the aforementioned values. In some embodiments, the arrays include a range of peptides ranging from about 10 to about 2.5M peptides. In some embodiments, the ncRNA peptides are in-situ synthesized on the array. In some embodiments, the ncRNA peptides are fixed on a substrate. In some embodiments, the substrate includes glass, silica, composite, resin, or combination thereof. In some embodiments, binding is detected by at least one of fluorescence, luminescence, calorimetry, chromatography, radioactivity, Bio-Layer Interferometry, electro-interference sensing, or surface plasmon resonance.

In some embodiments, the biological sample is selected from the group consisting of blood, plasma, serum, thymus, bone marrow, spleen, lymph node, bronchoalveolar lavage, breast, central nervous system, cerebrospinal fluid, eye, tears, gastrointestinal tract, saliva, feces, urine, heart, kidney, liver, lung, muscle, pancreas, peripheral nervous system, saliva, skin, thyroid, trachea, and tumor. In some embodiments, the cancer of interest is selected from the group consisting of acute lymphoblastic leukemia, acute monocytic leukemia, acute myeloid leukemia, acute promyelocytic leukemia, adenocarcinoma, adult T-cell leukemia, astrocytoma, bladder cancer, bone cancer, brain tumor, breast cancer, Burkitt's lymphoma, carcinoma, cervical cancer, chronic lymphocytic leukemia, chronic myelogenous leukemia, colon cancer, colorectal cancer, endometrial cancer, glioblastoma multiforme, glioma, hepatocellular carcinoma, Hodgkin's lymphoma, inflammatory breast cancer, kidney cancer, leukemia, lung cancer, lymphoma, malignant mesothelioma, medulloblastoma, melanoma, multiple myeloma, neuroblastoma, non-Hodgkin lymphoma, non-small cell lung cancer, ovarian cancer, pancreatic cancer, pituitary tumor, prostate cancer, retinoblastoma, skin cancer, small cell lung cancer, squamous cell carcinoma, stomach cancer, T-cell leukemia, T-cell lymphoma, thyroid cancer, and Wilms' tumor.

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the HIV Env-CD4-co-receptor complexes described herein.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994, and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody” or “antibodies” can also refer to a human antibody and/or a humanized antibody, where context so indicates. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

In embodiments, human antibodies can be prepared using any operable technique. Examples of techniques for human monoclonal antibody production include those described by Cole et al. (Monoclonal Antibodies and Cancer Therapy, Alan R. Liss, p. 77, 1985) and by Boemer et al. (J. Immunol., 147 (1): 86-95, 1991). Human antibodies (and fragments thereof) can also be produced using phage display libraries (Hoogenboom et al., J. Mol. Biol, 227:381, 1991; Marks et al., J Mol. Biol, 222:581, 1991).

Human antibodies can also be obtained from transgenic animals. For example, transgenic, mutant mice that are capable of producing a full repertoire of human antibodies, in response to immunization, have been described (see, e.g., Jakobovits et al., Proc. Natl Acad. ScL USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J (H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen), in some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human, in practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol, 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

Methods of producing a therapeutic or preventative antibody may be used in combination with other methods disclosed herein. For example, the array may include ncRNA peptides which have previously been identified as being immunogenic, cancer-specific, or shared across cancers according to the methods disclosed herein.

Note that as used herein and in the appended claims, the singular forms “a,” “an,” and “the” include plural referents unless the context clearly dictates otherwise. Thus, for example, reference to “an array” refers to one or more such arrays, and reference to “the method” includes reference to equivalent steps and methods known to those skilled in the art, and so forth.

It is contemplated that any embodied method or composition described herein can be implemented with respect to any other method or composition described herein.

As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%), 2%), 1%), or less in either direction (greater than or less than) of the stated reference value unless otherwise stated or otherwise evident from the context (except where such number would exceed 100% of a possible value).

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Although the embodiments are described in considerable detail with reference to certain methods and materials, one skilled in the art will appreciate that the disclosure herein can be practiced by other than the described embodiments, which have been presented for purposes of illustration and not of limitation. Therefore, the scope of the appended claims should not be limited to the description of the embodiments contained herein.

Certain embodiments of the invention are further described in the following example, which does not limit the scope of the invention described in the claims but rather is included to demonstrate such embodiments. It should be appreciated by those of skill in the art that the techniques disclosed in the examples which follow represent techniques developed by the inventors to function well in the practice of the methods provided herein, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1: ncRNA Peptides

Tables 1 and 2 below list 17 peptides (13 lncRNA and 4 pseudogene) that were predicted from the dark proteome, including a peptide sequence that was included in a peptide array and a corresponding neoantigen. The peptides were displayed on peptide arrays that were contacted with serum samples from 46 hemangiosarcoma (HSA)-diagnosed and 46 non-cancer dogs. The peptides listed in Tables 1 and 2 below were differentially bound by antibodies in serum samples from the HSA dogs versus non-cancer dogs.

The 17 dark proteome RNA-error derived neoantigens (REDNs) were identified by using the dog discovery peptide arrays (1.4 million peptides), which included REDNs from mis-transcription thru microsatellites, mis-initiation of translation of exon 1, mis-splicing of exons by excision or retention, mistranslation at tryptophan, and ncRNA (dark proteome), to evaluate 86 serum samples from 46 dogs diagnosed with stage 1 or 2 HSA and 46 dogs without cancer.

First, those REDN peptides with strong IgG antibody binding activity (>20,000 RFUs) on the arrays were identified for analysis. From these peptides, those that displayed a higher frequency of positives in the HSA dogs vs. non-cancer dogs were retained. A minimum positive differential of 6 dogs was set as threshold.

This assay was repeated, using the same sera on an independent dog discovery peptide array wafer and set of arrays. Results were obtained and the analysis repeated. Using these two datasets, peptides were identified that were differentially positive, by the same criteria, in both replicate assays. This identified 557 REDN peptides that were differentially positive in HSA diagnosed dogs, reproducibly.

The neoantigen source of these 557 REDN peptides was retrieved. Shown below are 17 of these REDNs which are ncRNA peptides and their corresponding neoantigen. Of these 17, 13 are lncRNAs and 4 are pseudogenes.

TABLE 1
Long non-coding RNA derived library peptides and their source
neoantigens

| ncRNA | |
|---|---|
| Peptide | ncRNA Neoantigen |
| AGWENQAG | AGWENQAGELKKILNTGPGSQELMVFSWARPLCWACVGTRTSR (SEQ |
| ELKKIL (SEQ | ID NO: 18) |
| ID NO: 1) | |
| SGPRPTCHL | SGPRPTCHLDRHSSLHIPPGPRHPQVNFLSYILLRVCVLMRGASA (SEQ |
| DRHSS (SEQ | ID NO: 19) |
| ID NO: 2) | |
| WKRQEEICP | THNEKEVNMQVETGVMWPQARRHLRPRSWKRQEEICPKAFGGGSMAL |
| KAFGG (SEQ | (SEQ ID NO: 20) |
| ID NO: 3) | |
| RGACCGAPA | RGACCGAPALGGAFLLLPPPTGRRKGVYEGSLRGSIRAAALWLPDVSES |
| LGGAF (SEQ | VFRLYTTSFFFFLIYL (SEQ ID NO: 21) |
| ID NO: 4) | |
| HRLSPQMTT | EFCYEEHNVHILHRLSPQMTTVQKAA (SEQ ID NO: 22) |
| VQKAA (SEQ | |
| ID NO: 5) | |
| KVRLSLSSRT | KVRLSLSSRTEGEAGAVRGVPGSTTPGLHPLIVTLSGTVP (SEQ ID NO: |
| EGEA (SEQ | 23) |
| ID NO: 6) | |
| LGNKPSYSE | QPRPARRVVPRETAAGAWGRALGRPCVSLGNKPSYSEEAGVRKDPPPL |
| EAGVR (SEQ | ELVVAGLPCDRSSSCPR (SEQ ID NO: 24) |
| ID NO: 7) | |
| WAGGSHMR | VGTGWTMQTCSWAGGSHMRTHMDIM (SEQ ID NO: 25) |
| THMDIM | |
| (SEQ ID NO: | |
| 8) | |
| WSRREGSGV | PGAGRRARWVTRRRGAHLSEPRRPPAKWWSRREGSGVPPPPPEDLGEA |
| PPPPP (SEQ | QDRPRTAARLLRKLKRVNVSEVRGAASVSL (SEQ ID NO: 26) |
| ID NO: 9) | |
| RGLPRPSCR | RGLPRPSCRDQA (SEQ ID NO: 27) |
| DQA (SEQ ID | |
| NO: 10) | |
| WAGDGFKIC | PVPGPYVPFSPVLLWAGDGFKICGQRVHLCSP (SEQ ID NO: 28) |
| GQRVH (SEQ | |
| ID NO: 11) | |
| WAGPVFSQR | SNKGKPPSGRQLRAWAGPVFSQRPPLGQRRVPS (SEQ ID NO: 29) |
| PPLGQ (SEQ | |
| ID NO: 12) | |
| WAPGSFCKD | YFLLIHAWAPGSFCKDSHSRS (SEQ ID NO: 30) |
| SHSRS (SEQ | |
| ID NO: 13) | |

TABLE 2
Pseudogene-derived library peptides and their source ncoantigens

| ncRNA Peptide | ncRNA Neoantigen |
|---|---|
| PRGPPFMDPWPVA | RAPSQSPRGPPFMDPWPVAA (SEQ ID NO: 31) |
| A (SEQ ID NO: 14) | |
| AGPGCASDYLQPSR | GGATGQRGMASSRAVGHGGQWGMVGSGAAGPGCASDYLQPSR |
| (SEQ ID NO: 15) | T (SEQ ID NO: 32) |
| CGTWGRPEREVSD | APGGAACGTWGRPEREVSDG (SEQ ID NO: 33) |
| G (SEQ ID NO: 16) | |
| MGPQACSAGLETA | GKPRGPVWGGRPWGPMAGGGRTALVPGDMGPQACSAGLETAT |
| T (SEQ ID NO: 17) | (SEQ ID NO: 34) |

### Example 2: Identification of Immunogenic, Cancer-Specific, or Shared ncRNAPs

To screen which ncRNAPs are immunogenic, cancer-specific, and/or shared across cancers, up to 5 million peptides are in-situ synthesized on a slide using mask-based photolithography in an Intel-like process. Blood samples are collected from subjects identified as having cancer or as being healthy/normal. The samples include the subject's antibodies. The samples are diluted in a suitable buffer. The samples are applied sequentially to the slide, and allowed to incubate. Binding of the samples to the peptides on the slide is measured and bioinformatically analyzed. Binding is compared between subjects with certain cancers, subjects with cancer generally, and subjects who do not have cancer. The analysis determines a smaller subset of ncRNAPs that are immunogenic, cancer-specific, and/or shared across cancers.

### Example 3: ncRNA Diagnostic Array

An array is created including peptides resulting from translation of a ncRNA (ncRNAP). The ncRNAPs on the array are spaced between 3 and 9 μm. A sample from a subject is applied to the array, and binding to the peptides of the array is measured. The sample is a blood sample, and the binding measured is binding of the subject's antibodies to the peptides of the array. The binding of the subject is compared to previously-measured binding patterns of other subjects who are known to have had cancer and other subjects who did not have cancer. Analysis of the binding predicts whether the subject has cancer.

### Example 4: ncRNA Predictive Array

An array is created including peptides resulting from translation of a ncRNA (ncRNAP). The ncRNAPs on the array are spaced between 3 and 9 μm apart. A sample from a subject with cancer is applied to the array, and binding of the sample to the peptides of the array is measured. The sample is a blood sample, and the binding measured is binding of the subject's antibodies to the peptides of the array. The binding of the subject is compared to previously-measured binding patterns of other subjects who are known to have had a positive response to immunotherapy (for example, immunotherapy was effective), and/or to other subjects who had an adverse reaction (e.g., an adverse event) in response to immunotherapy. Analysis of the binding predicts whether immunotherapy would be effective in treating the subject's cancer, and/or whether immunotherapy would provoke an adverse response in the subject. The immunotherapy is PD-1 inhibitor, a PD-L1 inhibitor, a CTLA-4 inhibitor, Pembrolizumab, Nivolumab, and/or Atezolizumab.

### Example 5: Personal ncRNA Vaccine

A biological sample (blood) is collected from an individual with cancer. The biological sample is applied to an array including ncRNA peptides. Binding of the individual's sample to the array is measured and analyzed. One or more peptides of the array are identified as being reactive with the individual's sample. A vaccine is designed including one or more peptides identified as being reactive with the patient's sample or including one or more nucleic acids encoding one or more peptides identified as being reactive with the patient's sample. The vaccine further includes an effective amount of an adjuvant. The vaccine is administered to the individual, and the vaccine elicits an immune response in the individual against the cancer. Administration of the vaccine causes reduction of tumor size, inhibition of tumor growth, reduction of tumor burden, increase of survival, and/or increase of cancer-free survival.

### Example 6: Vaccine Design

One or more ncRNAPs or one or more nucleic acids encoding ncRNAPs that are immunogenic, are identified. A vaccine is prepared including one or more ncRNAPs or one or more nucleic acids encoding ncRNAPs that are immunogenic. The vaccine also includes a suitable amount of a pharmaceutically acceptable carrier and/or adjuvant.

### Example 7: ncRNA Vaccine

A vaccine composition is created including ncRNAPs or nucleic acids encoding a ncRNAP. The vaccine further includes an effective amount of an adjuvant. The vaccine is administered to a subject for the prevention or treatment of cancer.

### Example 8: Therapeutic Molecule

A biological sample from a subject with cancer is contacted with an array including ncRNA peptides. Antibody binding to the array is detected, and one or more antibodies which bind a peptide of the array are selected. A therapeutic molecule including the one or more selected antibodies against a ncRNAPs is prepared and is administered to a subject with cancer in need thereof. The therapeutic molecule binds to a ncRNAP and improves the subject's immune response to the cancer.

In at least some of the previously described embodiments, one or more elements used in an embodiment can interchangeably be used in another embodiment unless such a replacement is not technically feasible. It will be appreciated by those skilled in the art that various other omissions, additions, and modifications may be made to the methods and structures described above without departing from the scope of the claimed subject matter. All such modifications and changes are intended to fall within the scope of the subject matter, as defined by the appended claims.

In addition, where features or aspects of the disclosure are described in terms of Markush groups, those skilled in the art will recognize that the disclosure is also thereby described in terms of any individual member or subgroup of members of the Markush group.

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those of skill in the art. The various aspects and embodiments disclosed herein are for purposes of illustration and are not intended to be limiting, with the true scope and spirit being indicated by the following claims.

What is claimed is:

1. A method of identifying one or more ncRNA peptides (ncRNAPs) that are immunogenic, cancer-specific, and/or shared across cancers, comprising:
a) contacting a ncRNA peptide array comprising a plurality of ncRNAPs with a first biological sample obtained from a first individual known to have a cancer,
b) measuring binding of the first biological sample to the ncRNA peptide array,
c) contacting the same type of ncRNA peptide array with a second biological sample obtained from a second individual,
d) measuring binding of the second biological sample to the ncRNA peptide array,
e) comparing the binding of the two biological samples to the ncRNA peptide array, and
f) identifying one or more ncRNAPs that are immunogenic, cancer-specific, and/or shared across cancers.

2. The method of claim 1, wherein measuring binding comprises detecting antibody reactivity to the plurality of ncRNAPs.

3. The method of claim 1, wherein the second individual is a control individual without cancer.

4. The method of claim 1, wherein the second individual is also known to have a cancer.

5. The method of claim 4, wherein the second individual is known to have the cancer of the first individual.

6. The method of claim 4, wherein the second individual and the first individual have different types of cancer.

7. The method of claim 1, further comprising determining a nucleic acid sequence of said ncRNAPs that are immunogenic to identify one or more nucleic acids encoding ncRNAPs that are immunogenic, cancer-specific, and/or shared across cancers.

8. The method of claim 1, wherein the ncRNA peptide array comprises at least about 10, about 100, about 200, about 300, about 500, about 1,000, about 2,000, about 3,000, about 5,000, about 7,500, about 10,000, about 12,500, about 15,000, about 17,500, about 20,000, about 22,500, about 25,000, about 27,500, about 30,000, about 32,500, about 35,000, about 37,500, about 40,000, about 50,000, about 100,000, about 200,000, about 300,000, about 400,000, about 500,000, about 600,000, about 700,000, about 800,000, about 900,000, about 1,000,000, about 1,500,000, about 2,000,000, about 2,500,000, about 3,000,000, about 3,500,000, about 4,000,000, about 4,500,000, or about 5,000,000 ncRNAPs.

9. The method of claim 1, wherein the plurality of ncRNAPs is in-situ synthesized on the array.

10. The method of claim 1, wherein the plurality of ncRNAPs is fixed on substrate.

11. The method of claim 10, wherein the substrate comprises glass, silica, composite, resin, or combination thereof.

12. The method of claim 1, wherein binding is detected by at least one of fluorescence, luminescence, calorimetry, chromatography, radioactivity, Bio-Layer Interferometry, electro-interference sensing, or surface plasmon resonance.

13. A method of measuring an immune response to a plurality of ncRNA peptides, the method comprising:
a) contacting a biological sample with an ncRNA peptide array comprising a plurality of ncRNA peptides,
b) measuring binding of the biological sample to the ncRNA peptide array.

14. The method of claim 13, wherein the ncRNA peptide array comprises at least one peptide having at least 95% sequence identity with any of SEQ ID NOs: 1-17.

15. The method of claim 13, wherein measuring binding comprises detecting antibody reactivity to the plurality of ncRNAPs.

16. The method of claim 13, wherein the ncRNA peptide array comprises at least about 10, about 100, about 200, about 300, about 500, about 1,000, about 2,000, about 3,000, about 5,000, about 7,500, about 10,000, about 12,500, about 15,000, about 17,500, about 20,000, about 22,500, about 25,000, about 27,500, about 30,000, about 32,500, about 35,000, about 37,500, about 40,000, about 50,000, about 100,000, about 200,000, about 300,000, about 400,000, about 500,000, about 600,000, about 700,000, about 800,000, about 900,000, about 1,000,000, about 1,500,000, about 2,000,000, about 2,500,000, about 3,000,000, about 3,500,000, about 4,000,000, about 4,500,000, or about 5,000,000 ncRNAPs.

17. The method of claim 13, wherein the plurality of ncRNAPs is in-situ synthesized on the array.

18. The method of claim 13, wherein the plurality of ncRNAPs is fixed on substrate.

19. The method of claim 18, wherein the substrate comprises glass, silica, composite, resin, or combination thereof.

20. The method of claim 13, wherein binding is detected by at least one of fluorescence, luminescence, calorimetry, chromatography, radioactivity, Bio-Layer Interferometry, electro-interference sensing, or surface plasmon resonance.

21. An array comprising a plurality of ncRNA peptides.

22. The array of claim 21, wherein the array comprises at least one peptide having at least 95% sequence identity with any of SEQ ID NOs: 1-17.

23. The array of claim 21, wherein the ncRNA peptides are spaced between 3 and 9 μm.

24. The array of claim 21, wherein the array comprises at least about 10, about 100, about 200, about 300, about 500, about 1,000, about 2,000, about 3,000, about 5,000, about 7,500, about 10,000, about 12,500, about 15,000, about 17,500, about 20,000, about 22,500, about 25,000, about 27,500, about 30,000, about 32,500, about 35,000, about 37,500, about 40,000, about 50,000, about 100,000, about 200,000, about 300,000, about 400,000, about 500,000, about 600,000, about 700,000, about 800,000, about 900,000, about 1,000,000, about 1,500,000, about 2,000,000, about 2,500,000, about 3,000,000, about 3,500,000, about 4,000,000, about 4,500,000, or about 5,000,000 ncRNAPs.

25. The array of claim 21, wherein the plurality of ncRNA peptides is in-situ synthesized on the array.

26. The array of claim 21, wherein the plurality of ncRNA peptides is fixed on a substrate.

27. The array of claim 26, wherein the substrate comprises glass, silica, composite, resin, or combination thereof.

28. The array of claim 21, wherein the array is configured to detect binding by at least one of fluorescence, luminescence, calorimetry, chromatography, radioactivity, Bio-Layer Interferometry, electro-interference sensing, or surface plasmon resonance.

29. A method of detecting cancer in a subject, the method comprising:
a) obtaining a biological sample from a subject,
b) contacting the biological sample with an array comprising a plurality of ncRNAPs;
c) measuring binding of the biological sample to the plurality of ncRNAPs; and
d) analyzing the binding to predict whether the subject has cancer.

30. The method of claim 29, wherein the array comprises at least one peptide having at least 95% sequence identity with any of SEQ ID NOs: 1-17.

31. The method of claim 29, wherein measuring binding comprises detecting antibody reactivity to at least one peptide of the array.

32. The method of claim 29, wherein the plurality of ncRNAPs is fixed on a substrate.

33. The method of claim 29, wherein the substrate comprises glass, silica, composite, resin, or combination thereof.

34. The method of claim 29, wherein the plurality of ncRNAPs is in-situ synthesized on the array.

35. The method of claim 29, wherein binding is detected by at least one of fluorescence, luminescence, calorimetry, chromatography, electro-interference, radioactivity, Bio-Layer Interferometry, electro-interference sensing, or surface plasmon resonance.

36. The method of claim 29, wherein the array comprises at least about 10, about 100, about 200, about 300, about 500, about 1,000, about 2,000, about 3,000, about 5,000, about 7,500, about 10,000, about 12,500, about 15,000, about 17,500, about 20,000, about 22,500, about 25,000, about 27,500, about 30,000, about 32,500, about 35,000, about 37,500, about 40,000, about 50,000, about 100,000, about 200,000, about 300,000, about 400,000, about 500,000, about 600,000, about 700,000, about 800,000, about 900,000, about 1,000,000, about 1,500,000, about 2,000,000, about 2,500,000, about 3,000,000, about 3,500,000, about 4,000,000, about 4,500,000, or about 5,000,000 ncRNAPs.

37. The method of any one of claims 21-27, wherein the biological sample comprises blood, serum, plasma, cerebrospinal fluid, saliva, urine, or combinations thereof.

38. The method of any one of claims 21-28, wherein the biological sample comprises antibodies.

39. The method of any one of claims 21-29, wherein the subject is a mammal.

40. The method of claim 30, wherein the subject is a human, a dog, a cat, a mouse, a rat, a rabbit, a horse, a cow, or a pig.

41. The method of any one of claims 21-31, wherein the subject is suspected of having a cancer.

42. The method of claim 32, wherein the cancer is selected from the group consisting of acute lymphoblastic leukemia, acute monocytic leukemia, acute myeloid leukemia, acute promyelocytic leukemia, adenocarcinoma, adult T-cell leukemia, astrocytoma, bladder cancer, bone cancer, brain tumor, breast cancer, Burkitt's lymphoma, carcinoma, cervical cancer, chronic lymphocytic leukemia, chronic myelogenous leukemia, colon cancer, colorectal cancer, endometrial cancer, glioblastoma multiforme, glioma, hepatocellular carcinoma, Hodgkin's lymphoma, inflammatory breast cancer, kidney cancer, leukemia, lung cancer, lymphoma, malignant mesothelioma, medulloblastoma, melanoma, multiple myeloma, neuroblastoma, non-Hodgkin lymphoma, non-small cell lung cancer, ovarian cancer, pancreatic cancer, pituitary tumor, prostate cancer, retinoblastoma, skin cancer, small cell lung cancer, squamous cell carcinoma, stomach cancer, T-cell leukemia, T-cell lymphoma, thyroid cancer, and Wilms' tumor.

43. A method of predicting the response of a subject with cancer to an immunotherapy, comprising
a) obtaining a biological sample from a subject with cancer,
b) contacting the biological sample to an array comprising a plurality of ncRNAPs, and
c) measuring binding of the biological sample to the plurality of ncRNAPs.

44. The method of claim 43, wherein the array comprises at least one peptide having at least 95% sequence identity with any of SEQ ID NOs: 1-17.

45. The method of claim 43, wherein measuring binding comprises detecting antibody reactivity to at least one peptide of the array.

46. The method of claim 43, further comprising analyzing the binding to predict whether immunotherapy would be effective in treating the subject's cancer.

47. The method of claim 43, further comprising analyzing the binding to predict whether immunotherapy would elicit an adverse immune response in the subject.

48. The method of 46, wherein analyzing the binding comprises comparing the binding of the biological sample to binding of another subject who responded positively to immunotherapy or experienced an adverse immune response in response to immunotherapy.

49. The method of claim 43, wherein the plurality of ncRNAPs is fixed on a substrate.

50. The method of claim 49, wherein the substrate comprises glass, silica, composite, resin, or combination thereof.

51. The method of claim 43, wherein the plurality of ncRNAPs is in-situ synthesized on the array.

52. The method of claim 43, wherein binding is detected by at least one of fluorescence, luminescence, calorimetry, chromatography, electro-interference, radioactivity, Bio-Layer Interferometry, electro-interference sensing, or surface plasmon resonance.

53. The method of claim 43, wherein the array comprises at least about 10, about 100, about 200, about 300, about 500, about 1,000, about 2,000, about 3,000, about 5,000, about 7,500, about 10,000, about 12,500, about 15,000, about 17,500, about 20,000, about 22,500, about 25,000, about 27,500, about 30,000, about 32,500, about 35,000, about 37,500, about 40,000, about 50,000, about 100,000, about 200,000, about 300,000, about 400,000, about 500,000, about 600,000, about 700,000, about 800,000, about 900,000, about 1,000,000, about 1,500,000, about 2,000,000, about 2,500,000, about 3,000,000, about 3,500,000, about 4,000,000, about 4,500,000, or about 5,000,000 ncRNAPs.

54. The method of claim 43, wherein the biological sample comprises blood, serum, plasma, cerebrospinal fluid, saliva, urine, or combinations thereof.

55. The method of claim 43, wherein the biological sample comprises antibodies.

56. The method of claim 43, wherein the subject is a mammal.

57. The method of claim 56, wherein the subject is a human, a dog, a cat, a mouse, a rat, a rabbit, a horse, a cow, or a pig.

58. The method of claim 43, wherein the cancer is selected from the group consisting of acute lymphoblastic leukemia, acute monocytic leukemia, acute myeloid leukemia, acute promyelocytic leukemia, adenocarcinoma, adult T-cell leukemia, astrocytoma, bladder cancer, bone cancer, brain tumor, breast cancer, Burkitt's lymphoma, carcinoma, cervical cancer, chronic lymphocytic leukemia, chronic myelogenous leukemia, colon cancer, colorectal cancer, endometrial cancer, glioblastoma multiforme, glioma, hepatocellular carcinoma, Hodgkin's lymphoma, inflammatory breast cancer, kidney cancer, leukemia, lung cancer, lymphoma, malignant mesothelioma, medulloblastoma, melanoma, multiple myeloma, neuroblastoma, non-Hodgkin lymphoma, non-small cell lung cancer, ovarian cancer, pancreatic cancer, pituitary tumor, prostate cancer, retinoblastoma, skin cancer, small cell lung cancer, squamous cell carcinoma, stomach cancer, T-cell leukemia, T-cell lymphoma, thyroid cancer, and Wilms' tumor.

59. A vaccine composition comprising one or more ncRNA peptides.

60. The vaccine composition of claim 59, further comprising an adjuvant.

61. The vaccine composition of claim 60, wherein the adjuvant is ABM2, AS01B, AS02, AS02A, Adjumer, Adjuvax, Algammulin, alum, aluminum phosphate, aluminum potassium sulfate, Bordetella pertussis, calcitriol, chitosan, cholcra toxin, CpG, dibutyl phthalatc, dimethyldioctadecylammonium bromide (DDA), Freund's adjuvant, Freund's complete, Freund's incomplete (IFA), GM-CSF, GMDP, gamma inulin, glycerol, HBSS (Hank's Balanced Salt Solution), polyinosinic-polycytidylic acid stabilized with polylysine and carboxymethylcellulose (poly-ICLC, also known as Hiltonol), IL-12, IL-2, imiquimod, interferon-gamma, ISCOM, lipid core peptide (LCP), lipofectin, lipopolysaccharide (LPS), liposomes, MF59, MLP+TDM, monophosphoryl lipid A, Montanide IMS-1313, Montanide ISA 206, Montanide ISA 720, Montanide ISA-51, Montanide ISA-50, nor-MDP, oil-in-water emulsion, P1005 (non-ionic copolymer), Pam3Cys (lipoprotein), pertussis toxin, poloxamer, QS21, RaLPS, Ribi, saponin, Seppic ISA 720, soybean oil, squalene, Syntex adjuvant formulation (SAF), synthetic polynucleotides (poly IC/poly AU), TiterMax tomatine, Vaxfectin, XtendIII, or Zymosan.

62. A method of designing a cancer vaccine, the method comprising:
a) identifying one or more ncRNAPs or one or more nucleic acids encoding ncRNAPs that are immunogenic, and
b) preparing a vaccine comprising one or more ncRNAPs or one or more nucleic acids encoding ncRNAPs that are immunogenic.

63. The method of claim 62, wherein identifying one or more ncRNAPs or one or more nucleic acids encoding ncRNAPs that are immunogenic comprises:
a) contacting a ncRNA peptide array comprising one or more ncRNAPs with a first biological sample obtained from a first individual known to have a cancer,
b) measuring binding of the first biological sample to the ncRNA peptide array,
c) contacting the same type of ncRNA peptide array with a second biological sample obtained from a second individual,
d) measuring binding of the second biological sample to the ncRNA peptide array,
e) comparing the binding of the two biological samples to the ncRNA peptide array, and
f) identifying one or more ncRNAPs that are immunogenic.

64. The method of claim 63, wherein the ncRNA peptide array comprises at least one peptide having at least 95% sequence identity with any of SEQ ID NOs: 1-17.

65. The method of 63, further comprising determining a nucleic acid sequence of said ncRNAPs that are immunogenic to identify one or more nucleic acids encoding ncRNAPs that are immunogenic.

66. The method of claim 63, wherein measuring binding comprises detecting antibody reactivity to the plurality of ncRNAPs.

67. The method of claim 63, wherein the second individual is a control individual without cancer.

68. The method of claim 63, wherein the second individual is also known to have a cancer.

69. The method of claim 68, wherein the second individual is known to have the cancer of the first individual.

70. The method of claim 68, wherein the second individual and the first individual have different types of cancer.

71. The method of claim 63, wherein the ncRNA peptide array comprises at least about 10, about 100, about 200, about 300, about 500, about 1,000, about 2,000, about 3,000, about 5,000, about 7,500, about 10,000, about 12,500, about 15,000, about 17,500, about 20,000, about 22,500, about 25,000, about 27,500, about 30,000, about 32,500, about 35,000, about 37,500, about 40,000, about 50,000, about 100,000, about 200,000, about 300,000, about 400,000, about 500,000, about 600,000, about 700,000, about 800,000, about 900,000, about 1,000,000, about 1,500,000, about 2,000,000, about 2,500,000, about 3,000,000, about 3,500,000, about 4,000,000, about 4,500,000, or about 5,000,000 ncRNAPs.

72. The method of claim 63, wherein the plurality of ncRNAPs is in-situ synthesized on the array.

73. The method of claim 63, wherein the plurality of ncRNAPs is fixed on substrate.

74. The method of claim 73-62, wherein the substrate comprises glass, silica, composite, resin, or combination thereof.

75. The method of claim 63, wherein binding is detected by at least one of fluorescence, luminescence, calorimetry, chromatography, radioactivity, Bio-Layer Interferometry, electro-interference sensing, or surface plasmon resonance.

76. The method of claim 62, wherein the vaccine further comprises an adjuvant.

77. The method of claim 76, wherein the adjuvant is ABM2, AS01B, AS02, AS02A, Adjumer, Adjuvax, Algammulin, alum, aluminum phosphate, aluminum potassium sulfate, Bordetella pertussis, calcitriol, chitosan, cholera toxin, CpG, dibutyl phthalate, dimethyldioctadecylammonium bromide (DDA), Freund's adjuvant, Freund's complete, Freund's incomplete (IFA), GM-CSF, GMDP, gamma inulin, glycerol, HBSS (Hank's Balanced Salt Solution), polyinosinic-polycytidylic acid stabilized with polylysine and carboxymethylcellulose (poly-ICLC, also known as Hiltonol), IL-12, IL-2, imiquimod, interferon-gamma, ISCOM, lipid core peptide (LCP), lipofectin, lipopolysaccharide (LPS), liposomes, MF59, MLP+TDM, monophosphoryl lipid A, Montanide IMS-1313, Montanide ISA 206, Montanide ISA 720, Montanide ISA-51, Montanide ISA-50, nor-MDP, oil-in-water emulsion, P1005 (non-ionic copolymer), Pam3Cys (lipoprotein), pertussis toxin, poloxamer, QS21, RaLPS, Ribi, saponin, Seppic ISA 720, soybean oil, squalene, Syntex adjuvant formulation (SAF), synthetic polynucleotides (poly IC/poly AU), TiterMax tomatine, Vaxfectin, XtendIII, or Zymosan.

78. A method of treating or preventing cancer, comprising administering a vaccine comprising a) one or more ncRNA peptides or b) a nucleic acid sequence encoding one or more ncRNA peptides.

79. The method of claim 78, wherein the vaccine further comprises an adjuvant.

80. The method of claim 79, wherein the adjuvant is ABM2, AS01B, AS02, AS02A, Adjumer, Adjuvax, Algammulin, alum, aluminum phosphate, aluminum potassium sulfate, Bordetella pertussis, calcitriol, chitosan, cholera toxin, CpG, dibutyl phthalate, dimethyldioctadecylammonium bromide (DDA), Freund's adjuvant, Freund's complete, Freund's incomplete (IFA), GM-CSF, GMDP, gamma inulin, glycerol, HBSS (Hank's Balanced Salt Solution), polyinosinic-polycytidylic acid stabilized with polylysine and carboxymethylcellulose (poly-ICLC, also known as Hiltonol), IL-12, IL-2, imiquimod, interferon-gamma, ISCOM, lipid core peptide (LCP), lipofectin, lipopolysaccharide (LPS), liposomes, MF59, MLP+TDM, monophosphoryl lipid A, Montanide IMS-1313, Montanide ISA 206, Montanide ISA 720, Montanide ISA-51, Montanide ISA-50, nor-MDP, oil-in-water emulsion, P1005 (non-ionic copolymer), Pam3Cys (lipoprotein), pertussis toxin, poloxamer, QS21, RaLPS, Ribi, saponin, Seppic ISA 720, soybean oil, squalene, Syntex adjuvant formulation (SAF), synthetic polynucleotides (poly IC/poly AU), TiterMax tomatine, Vaxfectin, XtendIII, or Zymosan.

81. The method of claim 78, wherein the vaccine is administered to a mammal.

82. The method of claim 81, wherein the vaccine is administered to a human, a dog, a cat, a mouse, a rat, a rabbit, a horse, a cow, or a pig.

83. The method of claim 78, wherein the cancer is selected from the group consisting of acute lymphoblastic leukemia, acute monocytic leukemia, acute myeloid leukemia, acute promyelocytic leukemia, adenocarcinoma, adult T-cell leukemia, astrocytoma, bladder cancer, bone cancer, brain tumor, breast cancer, Burkitt's lymphoma, carcinoma, cervical cancer, chronic lymphocytic leukemia, chronic myelogenous leukemia, colon cancer, colorectal cancer, endometrial cancer, glioblastoma multiforme, glioma, hepatocellular carcinoma, Hodgkin's lymphoma, inflammatory breast cancer, kidney cancer, leukemia, lung cancer, lymphoma, malignant mesothelioma, medulloblastoma, melanoma, multiple myeloma, neuroblastoma, non-Hodgkin lymphoma, non-small cell lung cancer, ovarian cancer, pancreatic cancer, pituitary tumor, prostate cancer, retinoblastoma, skin cancer, small cell lung cancer, squamous cell carcinoma, stomach cancer, T-cell leukemia, T-cell lymphoma, thyroid cancer, and Wilms' tumor.

84. The method of claim 78, wherein treating the cancer comprises reducing tumor size, inhibiting tumor growth, reducing tumor burden, increasing survival, or increasing cancer-free survival.

85. The method of claim 78, wherein administering the vaccine elicits an immune response in the subject against the cancer.

86. A method of treating a subject in need of treatment for a cancer, the method comprising:
a) obtaining a biological sample from the subject;
b) identifying one or more ncRNA peptides that are immunoreactive with a biological sample from the subject in a first population of ncRNA peptides;
c) preparing a vaccine composition comprising a second population of ncRNA peptides comprising one or more peptides identified in step b) or a nucleic acid sequence encoding the second population of peptides; and
d) administering an effective amount of the vaccine composition to the subject, thereby treating the cancer.

87. The method of claim 86, wherein identifying one or more ncRNA peptides that are immunoreactive comprises contacting the biological sample with an array comprising a first population of ncRNA peptides, measuring binding of the biological sample to the first population of ncRNA peptides of the array, and determining a second population of ncRNA peptides of the array that are immunoreactive with the biological sample.

88. The method of claim 87, wherein the first population of ncRNA peptides comprises at least one peptide having at least 95% sequence identity with any of SEQ ID NOs: 1-17.

89. The method of claim 87, wherein the second population of ncRNA peptides is a subpopulation of the first population of ncRNA peptides.

90. The method of claim 87, further comprising determining a nucleic acid sequence of said ncRNA peptides to identify one or more nucleic acids encoding ncRNA peptides that are immunogenic, cancer-specific, and/or shared across cancers.

91. The method of claim 86, wherein the biological sample is selected from the group consisting of blood, plasma, serum, thymus, bone marrow, spleen, lymph node, bronchoalveolar lavage, breast, central nervous system, cerebrospinal fluid, cyc, tears, gastrointestinal tract, saliva, feces, urine, heart, kidney, liver, lung, muscle, pancreas, peripheral nervous system, saliva, skin, thyroid, trachea, and tumor.

92. The method of claim 86, wherein the biological sample comprises an antibody.

93. The method of claim 86, wherein treating the cancer comprises reducing tumor size, inhibiting tumor growth, reducing tumor burden, increasing survival, or increasing cancer-free survival.

94. The method of claim 86, wherein administering the vaccine composition elicits an immune response in the subject against the cancer.

95. The method of claim 86, wherein the vaccine composition further comprises a pharmaceutically acceptable adjuvant or excipient.

96. The method of claim 95, wherein the adjuvant is ABM2, AS01B, AS02, AS02A, Adjumer, Adjuvax, Algammulin, alum, aluminum phosphate, aluminum potassium sulfate, Bordetella pertussis, calcitriol, chitosan, cholera toxin, CpG, dibutyl phthalate, dimethyldioctadecylammonium bromide (DDA), Freund's adjuvant, Freund's complete, Freund's incomplete (IFA), GM-CSF, GMDP, gamma inulin, glycerol, HBSS (Hank's Balanced Salt Solution), polyinosinic-polycytidylic acid stabilized with polylysine and carboxymethylcellulose (poly-ICLC, also known as Hiltonol), IL-12, IL-2, imiquimod, interferon-gamma, ISCOM, lipid core peptide (LCP), lipofectin, lipopolysaccharide (LPS), liposomes, MF59, MLP+TDM, monophosphoryl lipid A, Montanide IMS-1313, Montanide ISA 206, Montanide ISA 720, Montanide ISA-51, Montanide ISA-50, nor-MDP, oil-in-water emulsion, P1005 (non-ionic copolymer), Pam3Cys (lipoprotein), pertussis toxin, poloxamer, QS21, RaLPS, Ribi, saponin, Seppic ISA 720, soybean oil, squalene, Syntex adjuvant formulation (SAF), synthetic polynucleotides (poly IC/poly AU), TiterMax tomatine, Vaxfectin, XtendIII, or Zymosan.

97. The method of claim 86, wherein the cancer is selected from the group consisting of acute lymphoblastic leukemia, acute monocytic leukemia, acute myeloid leukemia, acute promyelocytic leukemia, adenocarcinoma, adult T-cell leukemia, astrocytoma, bladder cancer, bone cancer, brain tumor, breast cancer, Burkitt's lymphoma, carcinoma, cervical cancer, chronic lymphocytic leukemia, chronic myelogenous leukemia, colon cancer, colorectal cancer, endometrial cancer, glioblastoma multiforme, glioma, hepatocellular carcinoma, Hodgkin's lymphoma, inflammatory breast cancer, kidney cancer, leukemia, lung cancer, lymphoma, malignant mesothelioma, medulloblastoma, melanoma, multiple myeloma, neuroblastoma, non-Hodgkin lymphoma, non-small cell lung cancer, ovarian cancer, pancreatic cancer, pituitary tumor, prostate cancer, retinoblastoma, skin cancer, small cell lung cancer, squamous cell carcinoma, stomach cancer, T-cell leukemia, T-cell lymphoma, thyroid cancer, and Wilms' tumor.

98. The method of claim 86, wherein the subject is a mammal.

99. The method of claim 98, wherein the subject is a human, a dog, a cat, a mouse, a rat, a rabbit, a horse, a cow, or a pig.

100. A method of treating or preventing cancer, comprising administering a therapeutic molecule designed to bind an ncRNAP.

101. The method of claim 100, wherein the therapeutic molecule is an antibody or synthetic antibody.

102. The method of claim 100, wherein the therapeutic molecule is administered in combination with another cancer treatment.

103. The method of claim 100, wherein the cancer is selected from the group consisting of acute lymphoblastic leukemia, acute monocytic leukemia, acute myeloid leukemia, acute promyelocytic leukemia, adenocarcinoma, adult T-cell leukemia, astrocytoma, bladder cancer, bone cancer, brain tumor, breast cancer, Burkitt's lymphoma, carcinoma, cervical cancer, chronic lymphocytic leukemia, chronic myelogenous leukemia, colon cancer, colorectal cancer, endometrial cancer, glioblastoma multiforme, glioma, hepatocellular carcinoma, Hodgkin's lymphoma, inflammatory breast cancer, kidney cancer, leukemia, lung cancer, lymphoma, malignant mesothelioma, medulloblastoma, melanoma, multiple myeloma, neuroblastoma, non-Hodgkin lymphoma, non-small cell lung cancer, ovarian cancer, pancreatic cancer, pituitary tumor, prostate cancer, retinoblastoma, skin cancer, small cell lung cancer, squamous cell carcinoma, stomach cancer, T-cell leukemia, T-cell lymphoma, thyroid cancer, and Wilms' tumor.

104. The method of claim 100, wherein the therapeutic molecule is administered to a mammal.

105. The method of claim 104, wherein the therapeutic molecule is administered to a human, a dog, a cat, a mouse, a rat, a rabbit, a horse, a cow, or a pig.

106. The method of claim 100, wherein treating the cancer comprises reducing tumor size, inhibiting tumor growth, reducing tumor burden, increasing survival, or increasing cancer-free survival.

107. The method of claim 100, wherein administering the therapeutic molecule elicits an immune response in the subject against the cancer.

108. A method of producing a therapeutic or preventative antibody specific for cancer of interest, comprising:
a) contacting one or more biological samples obtained from one or more subjects identified as having the cancer of interest to an array comprising ncRNA peptides produced by one or more tumors;
b) detecting antibodies which bind to the ncRNA peptides;
c) selecting one or more ncRNA peptides that are immunoreactive with the antibodies; and
d) preparing an antibody composition against the selected ncRNA peptides for the cancer of interest.

109. The method of claim 108, wherein the array comprises at least one peptide having at least 95% sequence identity with any of SEQ ID NOs: 1-17.

110. The method of claim 108, wherein the ncRNA peptides of the array are spaced between 3 and 9 μm apart.

111. The method of claim 108, wherein the array comprises at least about 10, about 100, about 200, about 300, about 500, about 1,000, about 2,000, about 3,000, about 5,000, about 7,500, about 10,000, about 12,500, about 15,000, about 17,500, about 20,000, about 22,500, about 25,000, about 27,500, about 30,000, about 32,500, about 35,000, about 37,500, about 40,000, about 50,000, about 100,000, about 200,000, about 300,000, about 400,000, about 500,000, about 600,000, about 700,000, about 800,000, about 900,000, about 1,000,000, about 1,500,000, about 2,000,000, about 2,500,000, about 3,000,000, about 3,500,000, about 4,000,000, about 4,500,000, or about 5,000,000 ncRNAPs.

112. The method of claim 108, wherein the ncRNA peptides are in-situ synthesized on the array.

113. The method of claim 108, wherein the ncRNA peptides are fixed on a substrate.

114. The method of claim 113, wherein the substrate comprises glass, silica, composite, resin, or combination thereof.

115. The method of claim 108, wherein binding is detected by at least one of fluorescence, luminescence, calorimetry, chromatography, radioactivity, Bio-Layer Interferometry, electro-interference sensing, or surface plasmon resonance.

116. The method of claim 108, wherein the biological sample is selected from the group consisting of blood, plasma, serum, thymus, bone marrow, spleen, lymph node, bronchoalveolar lavage, breast, central nervous system, cerebrospinal fluid, eye, tears, gastrointestinal tract, saliva, feces, urine, heart, kidney, liver, lung, muscle, pancreas, peripheral nervous system, saliva, skin, thyroid, trachea, and tumor.

117. The method of claim 108, wherein the cancer of interest is selected from the group consisting of acute lymphoblastic leukemia, acute monocytic leukemia, acute myeloid leukemia, acute promyelocytic leukemia, adenocarcinoma, adult T-cell leukemia, astrocytoma, bladder cancer, bone cancer, brain tumor, breast cancer, Burkitt's lymphoma, carcinoma, cervical cancer, chronic lymphocytic leukemia, chronic myelogenous leukemia, colon cancer, colorectal cancer, endometrial cancer, glioblastoma multiforme, glioma, hepatocellular carcinoma, Hodgkin's lymphoma, inflammatory breast cancer, kidney cancer, leukemia, lung cancer, lymphoma, malignant mesothelioma, medulloblastoma, melanoma, multiple myeloma, neuroblastoma, non-Hodgkin lymphoma, non-small cell lung cancer, ovarian cancer, pancreatic cancer, pituitary tumor, prostate cancer, retinoblastoma, skin cancer, small cell lung cancer, squamous cell carcinoma, stomach cancer, T-cell leukemia, T-cell lymphoma, thyroid cancer, and Wilms' tumor.
